(19) United States
(12) Patent Application Publication
(10) Pub. No.: US 2007/0270970 A1
(43) Pub. Date: Nov. 22, 2007

(54) Spinal implants with improved wear resistance

(75) Inventor: Hai H. Trieu, Cordova, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/375,383

(22) Filed: Mar. 14, 2006

(51) Int. Cl. A61F 2/44

(52) U.S. Cl. 623/17.14

(57) ABSTRACT

An intervertebral prosthetic disc is disclosed and can be installed within an intervertebral space between a superior vertebra and an inferior vertebra. The intervertebral prosthetic disc can include an inferior component that can have a depression formed therein and a superior component that can have a projection extending therefrom. The projection can be configured to movably engage the depression and allow relative motion between the inferior component and the superior component. Further, the projection can include a superior wear resistant layer that can have a cross-linked polymer and can be configured to engage the depression.

### FIELD OF THE DISCLOSURE

The present disclosure relates generally to orthopedics and spinal surgery. More specifically, the present disclosure relates to spinal implants.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is a lateral view of a pair of adjacent vertebrae;

FIG. 3 is a top plan view of a vertebra;

FIG. 4 is a cross section view of an intervertebral disc;

FIG. 5 is an anterior view of a first embodiment of an intervertebral prosthetic disc;

FIG. 6 is an exploded anterior view of the first embodiment of the intervertebral prosthetic disc;

FIG. 7 is a cross-section view of the first embodiment of the intervertebral prosthetic disc;

FIG. 8 is a lateral view of the first embodiment of the intervertebral prosthetic disc;

FIG. 9 is an exploded lateral view of the first embodiment of the intervertebral prosthetic disc;

FIG. 10 is a plan view of a superior half of the first embodiment of the intervertebral prosthetic disc;

FIG. 11 is a plan view of an inferior half of the first embodiment of the intervertebral prosthetic disc;

FIG. 12 is an exploded lateral view of the first embodiment of the intervertebral prosthetic disc installed within an intervertebral space between a pair of adjacent vertebrae;

FIG. 13 is an anterior view of the first embodiment of the intervertebral prosthetic disc installed within an intervertebral space between a pair of adjacent vertrebrae;

FIG. 14 is a posterior view of a second embodiment of an intervertebral prosthetic disc;

FIG. 15 is an exploded posterior view of the second embodiment of the intervertebral prosthetic disc;

FIG. 16 is a cross-section view of the second embodiment of the intervertebral prosthetic disc;

FIG. 17 is a lateral view of the second embodiment of the intervertebral prosthetic disc;

FIG. 18 is an exploded lateral view of the second embodiment of the intervertebral prosthetic disc;

FIG. 19 is a plan view of a superior half of the second embodiment of the intervertebral prosthetic disc;

FIG. 20 is another plan view of the superior half of the second embodiment of the intervertebral prosthetic disc;

FIG. 21 is a plan view of an inferior half of the second embodiment of the intervertebral prosthetic disc;

FIG. 22 is another plan view of the inferior half of the second embodiment of the intervertebral prosthetic disc;

FIG. 23 is a lateral view of a third embodiment of an intervertebral prosthetic disc;

FIG. 24 is an exploded lateral view of the third embodiment of the intervertebral prosthetic disc;

FIG. 25 is a cross-section view of the third embodiment of the intervertebral prosthetic disc;

FIG. 26 is a anterior view of the third embodiment of the intervertebral prosthetic disc;

FIG. 27 is a perspective view of a superior component of the third embodiment of the intervertebral prosthetic disc;

FIG. 28 is a perspective view of an inferior component of the third embodiment of the intervertebral prosthetic disc;

FIG. 29 is a lateral view of a fourth embodiment of an intervertebral prosthetic disc;

FIG. 30 is an exploded lateral view of the fourth embodiment of the intervertebral prosthetic disc;

FIG. 31 is a cross-section view of the fourth embodiment of the intervertebral prosthetic disc;

FIG. 32 is a anterior view of the fourth embodiment of the intervertebral prosthetic disc;

FIG. 33 is a perspective view of a superior component of the fourth embodiment of the intervertebral prosthetic disc;

FIG. 34 is a perspective view of an inferior component of the fourth embodiment of the intervertebral prosthetic disc;

FIG. 35 is a posterior view of a fifth embodiment of an intervertebral prosthetic disc;

FIG. 36 is an exploded posterior view of the fifth embodiment of the intervertebral prosthetic disc;

FIG. 37 is a cross-section view of the fifth embodiment of the intervertebral prosthetic disc;

FIG. 38 is a plan view of a superior half of the fifth embodiment of the intervertebral prosthetic disc;

FIG. 39 is a plan view of an inferior half of the fifth embodiment of the intervertebral prosthetic disc;

FIG. 40 is a perspective view of a sixth embodiment of an intervertebral prosthetic disc;

FIG. 41 is a superior plan view of the sixth embodiment of the intervertebral prosthetic disc;

FIG. 42 is an anterior plan view of the sixth embodiment of the intervertebral prosthetic disc;

FIG. 43 is a cross-section view of the sixth embodiment of the intervertebral prosthetic disc taken along line 43-43 in FIG. 41;

FIG. 44 is a plan view of a nucleus implant installed within an intervertebral disc;

FIG. 45 is a plan view of the nucleus implant within a nucleus delivery device;

FIG. 46 is a plan view of the nucleus implant exiting the nucleus delivery device; and

FIG. 47 is a cross-section view of the nucleus implant.

### DETAILED DESCRIPTION OF THE DRAWINGS

An intervertebral prosthetic disc is disclosed and can be installed within an intervertebral space between a superior vertebra and an inferior vertebra. The intervertebral prosthetic disc can include an inferior component that can have a depression formed therein and a superior component that can have a projection extending therefrom. The projection can be configured to movably engage the depression and allow relative motion between the inferior component and the superior component. Further, the projection can include a superior wear resistant layer that can have a cross-linked polymer and can be configured to engage the depression.

In another embodiment, an intervertebral prosthetic disc is disclosed and can be installed within an intervertebral space between a superior vertebra and an inferior vertebra. The intervertebral prosthetic disc can include an inferior component that can have an inferior depression formed therein and a superior component having a superior depression formed therein. Additionally, a nucleus can be disposed between the inferior component and the superior component. The nucleus can include a superior wear resistant layer and an inferior wear resistant layer. The superior wear resistant layer of the nucleus can be a cross-linked polymer and can be configured to movably engage the superior depression. Also, the inferior wear resistant layer of the nucleus can be configured to movably engage the inferior depression.

In yet another embodiment, an intervertebral prosthetic disc is disclosed and can be installed within an intervertebral space between a superior vertebra and an inferior vertebra. The intervertebral prosthetic disc can include an inferior component that can have an inferior projection extending therefrom and a superior component that can have a superior projection extending therefrom. A nucleus can be disposed between the inferior component and the superior component. The nucleus can include a superior depression that can have a superior wear resistant layer therein and an inferior depression that can have an inferior wear resistant layer therein. Further, the superior wear resistant layer of the nucleus can be a cross-linked polymer and can be configured to movably engage the superior projection. The inferior wear resistant layer of the nucleus can be configured to movably engage the inferior projection.

In still yet another embodiment, an intervertebral prosthetic disc is disclosed and can be installed within an intervertebral space between a superior vertebra and an inferior vertebra. The intervertebral prosthetic disc can include an inferior component, a superior component, and a generally toroidal nucleus that can be disposed between the inferior component and the superior component. The nucleus can include a core and an outer wear resistant layer on the core. The outer wear resistant layer of the core can be a cross-linked polymer and can be configured to movably engage the inferior component and the superior component.

In yet still another embodiment, a nucleus implant is disclosed and can be installed within an intervertebral space within an intervertebral disc. The nucleus implant can include a load bearing elastic body that can be movable between a folded configuration and a substantially straight configuration. The load bearing elastic body can have a core and an outer wear resistant layer around the core. Moreover, the outer wear resistant layer can be a cross-linked polymer.

In another embodiment, an intervertebral prosthetic disc is disclosed and can be installed within an intervertebral space between a superior vertebra and an inferior vertebra. The intervertebral prosthetic disc can include a first polymer component having a main body and a wear surface, wherein the wear surface exhibits a higher degree of cross-linking than a portion of the main body.

In still another embodiment, an intervention kit for field use is disclosed and can include an intervertebral prosthetic disc comprising a polymer and a cross-linking agent.

In yet another embodiment, a method of implanting an intervertebral prosthetic disc within an intervertebral space is disclosed and can include exposing the intervertebral prosthetic disc to a cross-linking agent and positioning the intervertebral prosthetic disc within the intervertebral space.

In another embodiment, a method of implanting an intervertebral prosthetic disc within an intervertebral space is disclosed and can include positioning the intervertebral prosthetic disc within the intervertebral space and exposing the intervertebral prosthetic disc to a cross-linking agent.

In still another embodiment, a spinal implant is disclosed and can be installed between a superior vertebra and an inferior vertebra. The spinal implant can include a polymeric component having a surface. Further, the surface of the polymeric core can be cross-linked greater than an underlying material.

Description of Relevant Anatomy

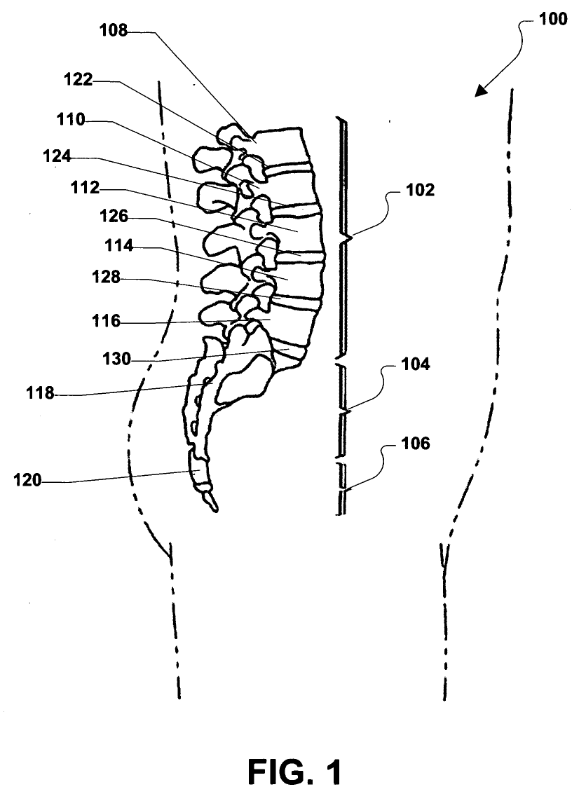

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

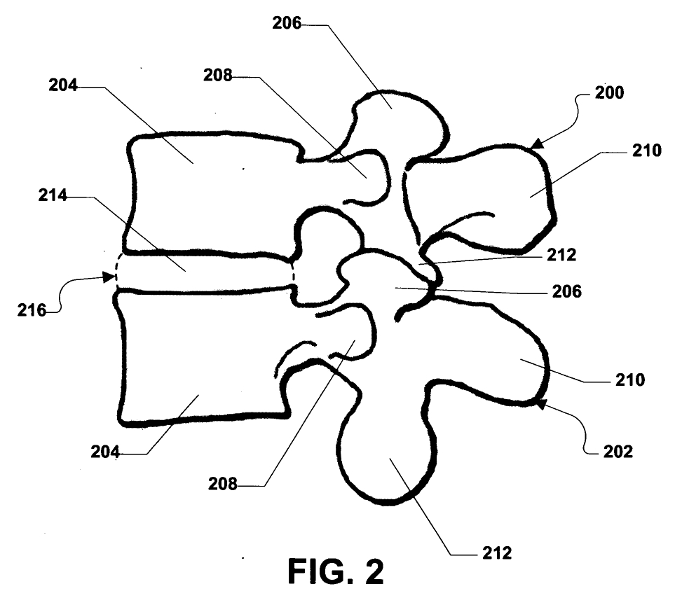

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

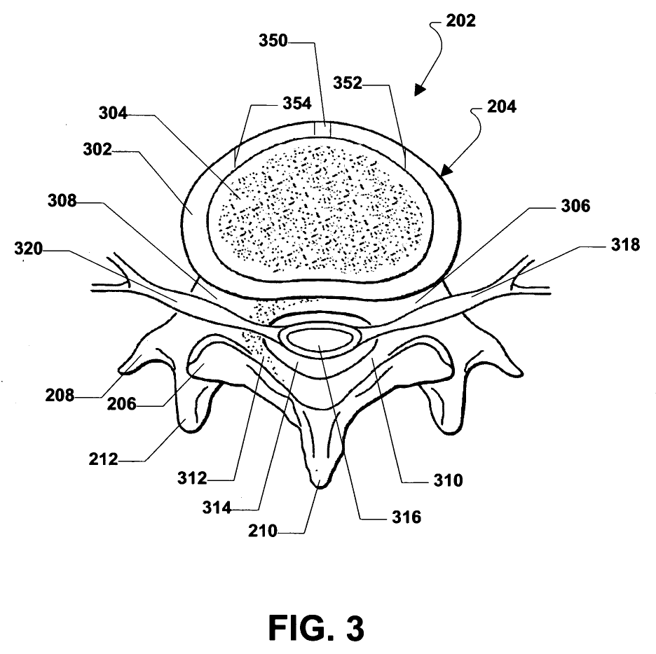

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel cutting device, e.g., a keel chisel designed to cut a groove in a vertebra, prior to the installation of the intervertebral prosthetic disc. Further, the keel groove 350 is sized and shaped to receive and engage a keel, described in detail below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

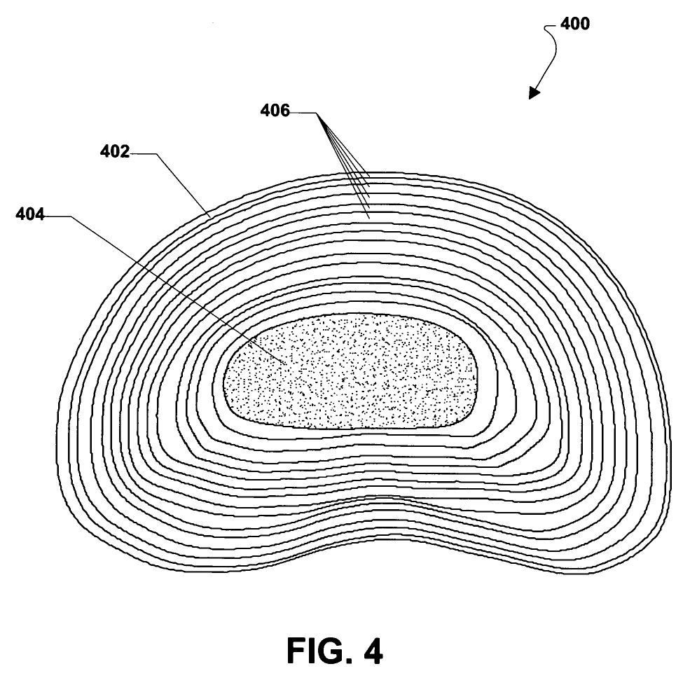

Referring now to FIG. 4, an intervertebral disc is shown and is generally designated 400. The intervertebral disc 400 is made up of two components: the annulus fibrosis 402 and the nucleus pulposus 404. The annulus fibrosis 402 is the outer portion of the intervertebral disc 400, and the annulus fibrosis 402 includes a plurality of lamellae 406. The lamellae 406 are layers of collagen and proteins. Each lamella 406 includes fibers that slant at 30-degree angles, and the fibers of each lamella 406 run in a direction opposite the adjacent layers. Accordingly, the annulus fibrosis 402 is a structure that is exceptionally strong, yet extremely flexible.

The nucleus pulposus 404 is the inner gel material that is surrounded by the annulus fibrosis 402. It makes up about forty percent (40%) of the intervertebral disc 400 by weight. Moreover, the nucleus pulposus 404 can be considered a ball-like gel that is contained within the lamellae 406. The nucleus pulposus 404 includes loose collagen fibers, water, and proteins. The water content of the nucleus pulposus 404 is about ninety percent (90%) by weight at birth and decreases to about seventy percent by weight (70%) by the fifth decade.

Injury or aging of the annulus fibrosis 402 may allow the nucleus pulposus 404 to be squeezed through the annulus fibers either partially, causing the disc to bulge, or completely, allowing the disc material to escape the intervertebral disc 400. The bulging disc or nucleus material may compress the nerves or spinal cord, causing pain. Accordingly, the nucleus pulposus 404 can be removed and replaced with an artificial nucleus.

### DESCRIPTION OF A FIRST EMBODIMENT OF AN INTERVERTEBRAL PROSTHETIC DISC

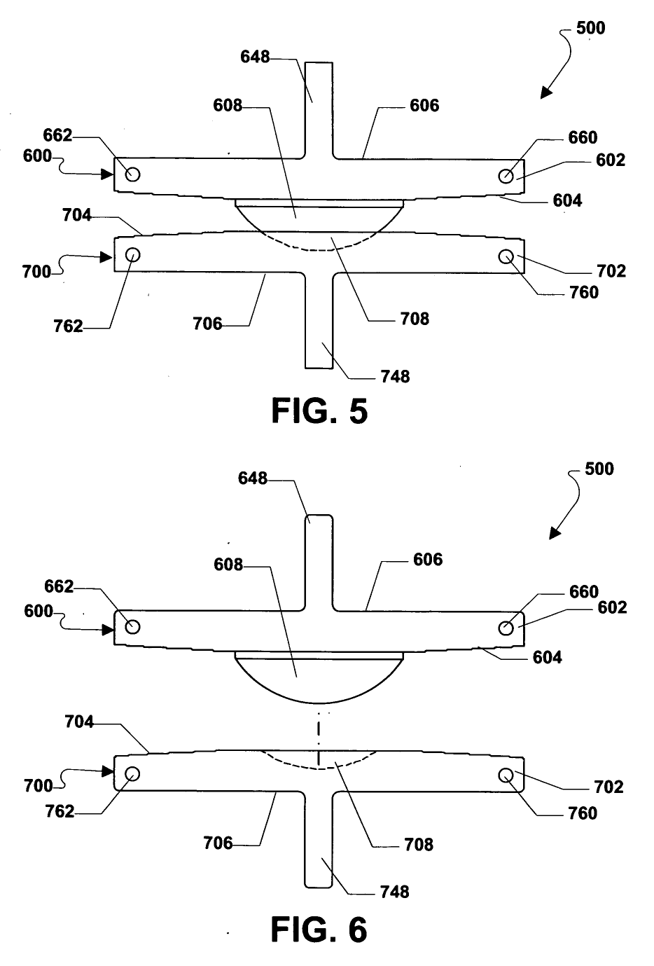

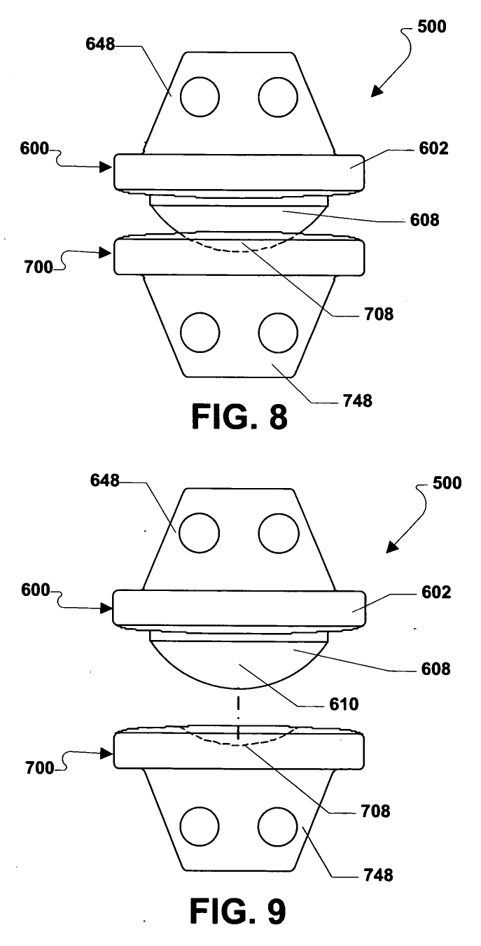

Referring to FIGS. 5 through 11 a first embodiment of an intervertebral prosthetic disc is shown and is generally designated 500. As illustrated, the intervertebral prosthetic disc 500 can include a superior component 600 and an inferior component 700. In a particular embodiment, the components 600, 700 can be made from one or more biocompatible materials. For example, the biocompatible materials can be one or more polymer materials.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, fluoropolyolefin, polybutadiene, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 600, 700 can be made from any other substantially rigid biocompatible materials.

In a particular embodiment, the superior component 600 can include a superior support plate 602 that has a superior articular surface 604 and a superior bearing surface 606. In a particular embodiment, the superior articular surface 604 can be generally curved and the superior bearing surface 606 can be substantially flat. In an alternative embodiment, the superior articular surface 604 can be substantially flat and at least a portion of the superior bearing surface 606 can be generally curved.

As illustrated in FIG. 5 through FIG. 9, a projection 608 extends from the superior articular surface 604 of the superior support plate 602. In a particular embodiment, the projection 608 has a hemi-spherical shape. Alternatively, the projection 608 can have an elliptical shape, a cylindrical shape, or other arcuate shape.

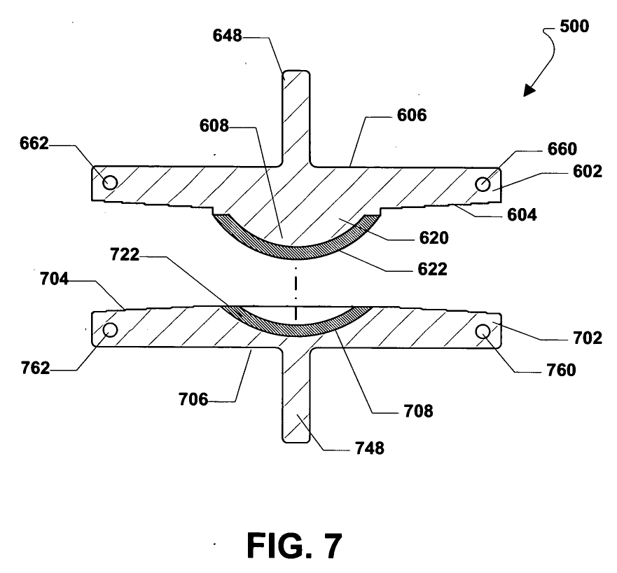

Referring to FIG. 7, the projection 608 can include a superior wear resistant layer 622. In a particular embodiment, the superior wear resistant layer 622 can be formed by cross-linking the surface of the projection 608. In a particular embodiment, depending on the type of material of which the projection 608 is comprised, the surface of the projection 608 can be cross-linked using a cross-linking agent. Acceptable cross-linking agents can include heat (thermal energy), various spectra or wavelengths of light, moisture, chemical agents/reagents, a radiation source (e.g., a thermal radiation source, a light radiation source, or another radiation source), or any combination of cross-linking agents. Further, the surface of the projection 608 can be cross-linked by exposing the surface of the projection 608 to a cross-linking agent in the presence of a catalyst that promotes cross-linking in the subject material. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the projection 608 can be cross-linked to a depth of about five millimeters (5 mm) or less, such as about three millimeters (3 mm) or less. In this manner, the material underlying the wear resistant layer 622 can exhibit the typical material properties associated with the uncross-linked material that comprises the projection 608.

Accordingly, the hardness of the wear resistant layer 622 can be greater than the hardness of the underlying material. Further, the Young's modulus of the wear resistant layer 622 can be greater than the Young's modulus of the underlying material. Also, the toughness of the wear resistant layer 622 can be greater than the toughness of the underlying material.

Further, in a particular embodiment, the surface of the projection 608 can be cross-linked in such a fashion that the hardness of the wear resistant layer 622 decreases from a maximum at or near the surface of the wear resistant layer 622 to the underlying uncross-linked material of the projection 608. This can create a hardness gradient that substantially minimizes or eliminates an extreme change in hardness between the wear resistant layer 622 and the projection 608. Further, the gradual change of the hardness gradient can substantially minimize or eliminate the chance that the wear resistant layer 622 may delaminate from the projection 608.

In another particular embodiment, the underlying material of the projection 608 may be cross-linked. However, in such a case, the mean or average cross-linking of the wear resistant layer 622 may be greater than the underlying cross-linked material.

The cross-linking agent can be introduced or applied at various points during manufacture of the prosthetic disc in order to accommodate various manufacturing parameters, including the desired degree of cross-linking at or near the surface. Alternatively, the cross-linking agent can be introduced or applied post-manufacture, yet prior to implantation (e.g., by surgical staff or the like). Alternatively, in certain embodiments, the cross-linking agent can be introduced or applied after implantation. Further, a cross-linking agent can be introduced or applied at various points between the beginning of manufacture and the end of the implantation procedure. Two or more different cross-linking agents can be introduced or applied at various points, as desired, to obtain the proper degree of cross-linking in the desired location(s). The cross-linking agent(s) can be provided along with all or a portion of the prosthetic disc in kit form for ease of use in the field.

FIG. 5 through FIG. 9 indicate that the superior component 600 can include a superior keel 648 that extends from superior bearing surface 606. During installation, described below, the superior keel 648 can at least partially engage a keel groove that can be established within a cortical rim of a vertebra. Further, the superior keel 648 can be coated with a bone-growth promoting substance, e.g., a hydroxyapatite coating formed of calcium phosphate. Additionally, the superior bearing surface 606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

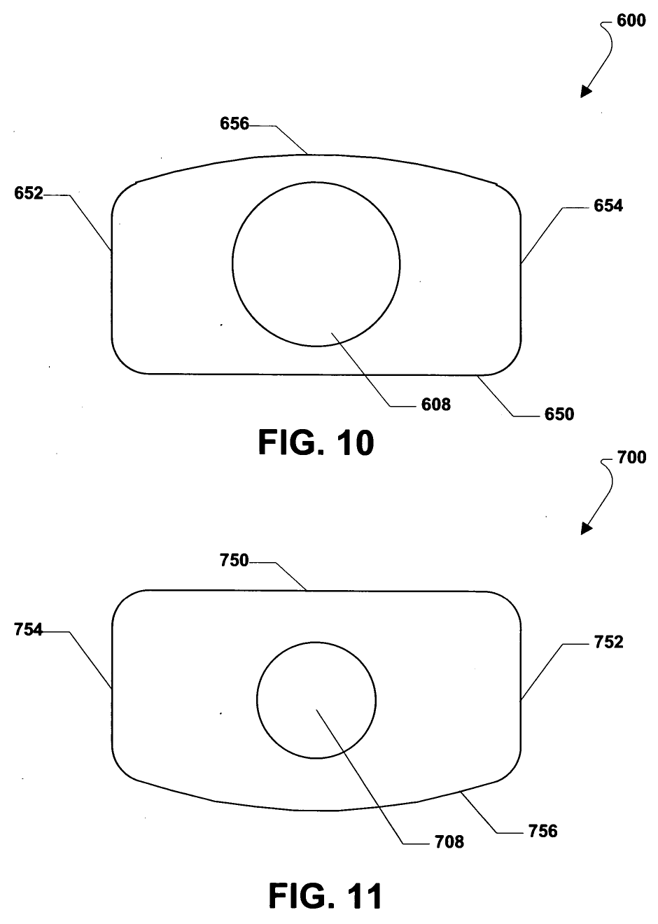

As illustrated in FIG. 10, the superior component 600 can be generally rectangular in shape. For example, the superior component 600 can have a substantially straight posterior side 650. A first straight lateral side 652 and a second substantially straight lateral side 654 can extend substantially perpendicular from the posterior side 650 to an anterior side 656. In a particular embodiment, the anterior side 656 can curve outward such that the superior component 600 is wider through the middle than along the lateral sides 652, 654. Further, in a particular embodiment, the lateral sides 652, 654 are substantially the same length.

FIG. 5 through FIG. 7 show that the superior component 600 can include a first implant inserter engagement hole 660 and a second implant inserter engagement hole 662. In a particular embodiment, the implant inserter engagement holes 660, 662 are configured to receive respective dowels, or pins, that extend from an implant inserter (not shown) that can be used to facilitate the proper installation of an intervertebral prosthetic disc, e.g., the intervertebral prosthetic disc 500 shown in FIG. 5 through FIG. 11.

In a particular embodiment, the inferior component 700 can include an inferior support plate 702 that has an inferior articular surface 704 and an inferior bearing surface 706. In a particular embodiment, the inferior articular surface 704 can be generally curved and the inferior bearing surface 706 can be substantially flat. In an alternative embodiment, the inferior articular surface 704 can be substantially flat and at least a portion of the inferior bearing surface 706 can be generally curved.

As illustrated in FIG. 5 through FIG. 9, a depression 708 extends into the inferior articular surface 704 of the inferior support plate 702. In a particular embodiment, the depression 708 is sized and shaped to receive the projection 608 of the superior component 600. For example, the depression 708 can have a hemi-spherical shape. Alternatively, the depression 708 can have an elliptical shape, a cylindrical shape, or other arcuate shape.

Referring to FIG. 7, the depression 708 can include an inferior wear resistant layer 722. In a particular embodiment, the inferior wear resistant layer 722 can be formed by cross-linking the surface of the depression 708. In a particular embodiment, depending on the type of material of which the depression 708 is comprised, the surface of the depression 708 can be cross-linked using a cross-linking agent. Acceptable cross-linking agents can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the depression 708 can be cross-linked by exposing the surface of the depression 708 to a radiation source in the presence of a catalyst that promotes cross-linking in the subject material. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the depression 708 can be cross-linked to a depth of about five millimeters (5 mm) or less, such as about three millimeters (3 mm) or less. In this manner, the material underlying the wear resistant layer 722 can exhibit the typical material properties associated with the uncross-linked material that comprises the depression 708.

Accordingly, the hardness of the wear resistant layer 722 can be greater than the hardness of the underlying material. Further, the Young's modulus of the wear resistant layer 722 can be greater than the Young's modulus of the underlying material. Also, the toughness of the wear resistant layer 722 can be greater than the toughness of the underlying material.

Further, in a particular embodiment, the surface of the depression 708 can be cross-linked in such a fashion that the hardness of the wear resistant layer 722 decreases from a maximum at or near the surface of the wear resistant layer 722 to the underlying uncross-linked material of the depression 708. This can create a hardness gradient that substantially minimizes or eliminates an extreme change in hardness between the wear resistant layer 722 and the depression 708. Further, the hardness gradient substantially minimizes or eliminates the chance that the wear resistant layer 722 may delaminate from the depression 708.

In another particular embodiment, the underlying material of the depression 708 may be cross-linked. However, in such a case, the mean or average cross-linking of the wear resistant layer 722 may be greater than the underlying cross-linked material.

FIG. 5 through FIG. 9 indicate that the inferior component 700 can include an inferior keel 748 that extends from inferior bearing surface 706. During installation, described below, the inferior keel 748 can at least partially engage a keel groove that can be established within a cortical rim of a vertebra, e.g., the keel groove 350 shown in FIG. 3. Further, the inferior keel 748 can be coated with a bone-growth promoting substance, e.g., a hydroxyapatite coating formed of calcium phosphate. Additionally, the inferior bearing surface 706 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

In a particular embodiment, as shown in FIG. 11, the inferior component 700 can be shaped to match the shape of the superior component 600, shown in FIG. 10. Further, the inferior component 700 can be generally rectangular in shape. For example, the inferior component 700 can have a substantially straight posterior side 750. A first straight lateral side 752 and a second substantially straight lateral side 754 can extend substantially perpendicular from the posterior side 750 to an anterior side 756. In a particular embodiment, the anterior side 756 can curve outward such that the inferior component 700 is wider through the middle than along the lateral sides 752, 754. Further, in a particular embodiment, the lateral sides 752, 754 are substantially the same length.

FIG. 5 through FIG. 7 show that the inferior component 700 can include a first implant inserter engagement hole 760 and a second implant inserter engagement hole 762. In a particular embodiment, the implant inserter engagement holes 760, 762 are configured to receive respective dowels, or pins, that extend from an implant inserter (not shown) that can be used to facilitate the proper installation of an intervertebral prosthetic disc, e.g., the intervertebral prosthetic disc 500 shown in FIG. 5 through FIG. 11.

In a particular embodiment, the overall height of the intervertebral prosthetic device 500 can be in a range from fourteen millimeters to forty-six millimeters (14-46 mm). Further, the installed height of the intervertebral prosthetic device 500 can be in a range from eight millimeters to sixteen millimeters (8-16 mm). In a particular embodiment, the installed height can be substantially equivalent to the distance between an inferior vertebra and a superior vertebra when the intervertebral prosthetic device 500 is installed there between.

In a particular embodiment, the length of the intervertebral prosthetic device 500, e.g., along a longitudinal axis, can be in a range from thirty millimeters to forty millimeters (30-40 mm). Additionally, the width of the intervertebral prosthetic device 500, e.g.; along a lateral axis, can be in a range from twenty-five millimeters to forty millimeters (25-40 mm). Moreover, in a particular embodiment, each keel 648, 748 can have a height in a range from three millimeters to fifteen millimeters (3-15 mm).

### INSTALLATION OF THE FIRST EMBODIMENT WITHIN AN INTERVERTEBRAL SPACE

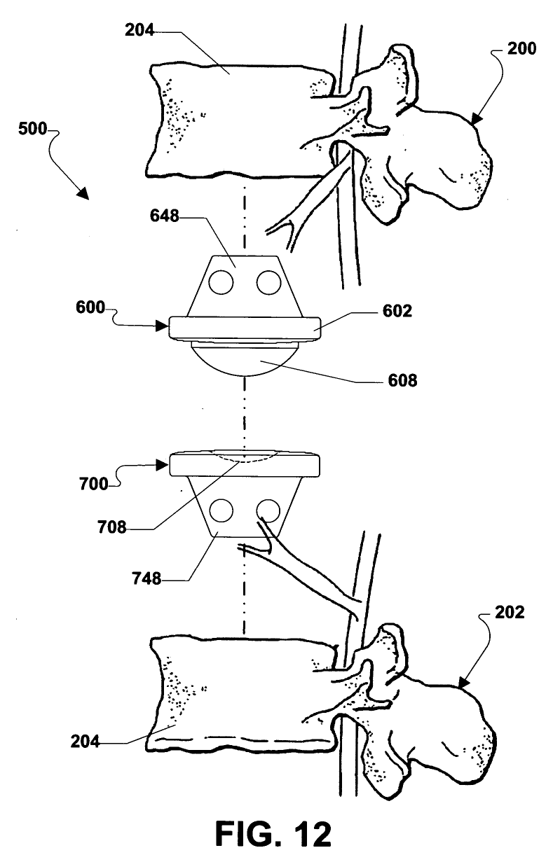

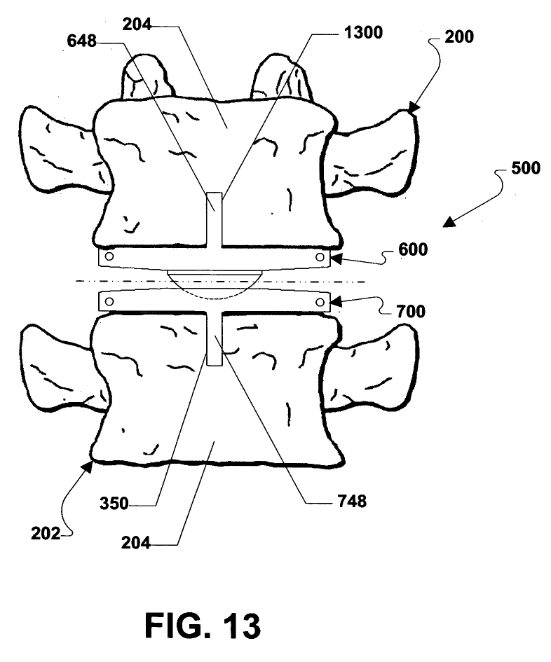

Referring to FIG. 12 and FIG. 13, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 500 described in conjunction with FIG. 5 through FIG. 11. Alternatively, the intervertebral prosthetic disc can be an intervertebral prosthetic disc according to any of the embodiments disclosed herein.

As shown in FIG. 12 and FIG. 13, the intervertebral prosthetic disc 500 is installed within the intervertebral space 214 that can be established between the superior vertebra 200 and the inferior vertebra 202 by removing vertebral disc material (not shown). FIG. 13 shows that the superior keel 648 of the superior component 600 can at least partially engage the cancellous bone and cortical rim of the superior vertebra 200. Further, as shown in FIG. 13, the superior keel 648 of the superior component 600 can at least partially engage a superior keel groove 1300 that can be established within the vertebral body 204 of the superior vertebra 202. In a particular embodiment, the vertebral body 204 can be further cut to allow the superior support plate 602 of the superior component 600 to be at least partially recessed into the vertebral body 204 of the superior vertebra 200.

Also, as shown in FIG. 12, the inferior keel 748 of the inferior component 700 can at least partially engage the cancellous bone and cortical rim of the inferior vertebra 202. Further, as shown in FIG. 13, the inferior keel 748 of the inferior component 700 can at least partially engage the inferior keel groove 350, previously introduced and described in conjunction with FIG. 3, which can be established within the vertebral body 204 of the inferior vertebra 202. In a particular embodiment, the vertebral body 204 can be further cut to allow the inferior support plate 702 of the inferior component 700 to be at least partially recessed into the vertebral body 204 of the inferior vertebra 200.

As illustrated in FIG. 12 and FIG. 13, the projection 608 that extends from the superior component 600 of the intervertebral prosthetic disc 500 can at least partially engage the depression 708 that is formed within the inferior component 700 of the intervertebral prosthetic disc 500. More specifically, the superior wear resistant layer 622 of the superior component 600 can at least partially engage the inferior wear resistant layer 722 of the inferior component 700. Further, the superior wear resistant layer 622 of the superior component 600 can movably engage the inferior wear resistant layer 722 of the inferior component 700 to allow relative motion between the superior component 600 and the inferior component 700.

It is to be appreciated that when the intervertebral prosthetic disc 500 is installed between the superior vertebra 200 and the inferior vertebra 202, the intervertebral prosthetic disc 500 allows relative motion between the superior vertebra 200 and the inferior vertebra 202. Specifically, the configuration of the superior component 600 and the inferior component 700 allows the superior component 600 to rotate with respect to the inferior component 700. As such, the superior vertebra 200 can rotate with respect to the inferior vertebra 202.

In a particular embodiment, the intervertebral prosthetic disc 500 can allow angular movement in any radial direction relative to the intervertebral prosthetic disc 500.

Further, as depicted in FIG. 11 through 13, the inferior component 700 can be placed on the inferior vertebra 202 so that the center of rotation of the inferior component 700 is substantially aligned with the center of rotation of the inferior vertebra 202. Similarly, the superior component 600 can be placed relative to the superior vertebra 200 so that the center of rotation of the superior component 600 is substantially aligned with the center of rotation of the superior vertebra 200. Accordingly, when the vertebral disc, between the inferior vertebra 202 and the superior vertebra 200, is removed and replaced with the intervertebral prosthetic disc 500 the relative motion of the vertebrae 200, 202 provided by the vertebral disc is substantially replicated.

### DESCRIPTION OF A SECOND EMBODIMENT OF AN INTERVERTEBRAL PROSTHETIC DISC

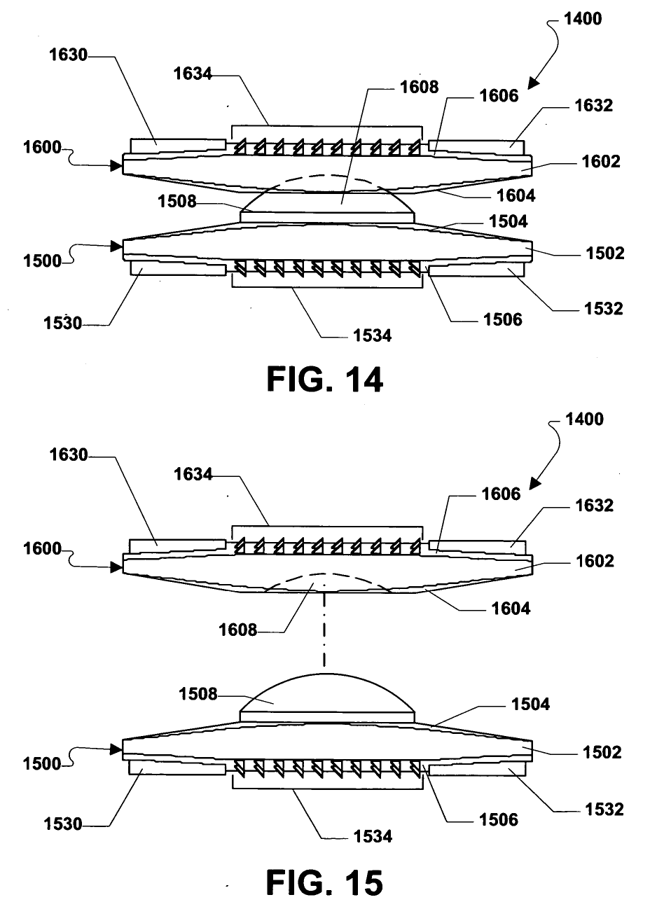

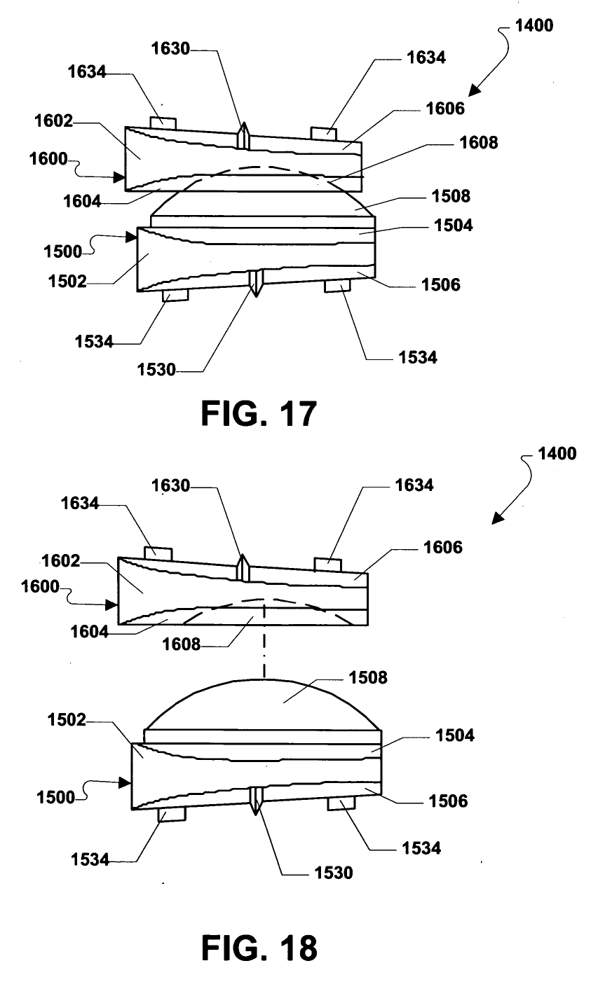

Referring to FIGS. 14 through 22 a second embodiment of an intervertebral prosthetic disc is shown and is generally designated 1400. As illustrated, the intervertebral prosthetic disc 1400 can include an inferior component 1500 and a superior component 1600. In a particular embodiment, the components 1500, 1600 can be made from one or more biocompatible materials. For example, the biocompatible materials can be one or more polymer materials.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, fluoropolyolefin, polybutadiene, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 1500, 1600 can be made from any other substantially rigid biocompatible materials.

In a particular embodiment, the inferior component 1500 can include an inferior support plate 1502 that has an inferior articular surface 1504 and an inferior bearing surface 1506. In a particular embodiment, the inferior articular surface 1504 can be generally rounded and the inferior bearing surface 1506 can be generally flat.

As illustrated in FIG. 14 through FIG. 22, a projection 1508 extends from the inferior articular surface 1504 of the inferior support plate 1502. In a particular embodiment, the projection 1508 has a hemi-spherical shape. Alternatively, the projection 1508 can have an elliptical shape, a cylindrical shape, or other arcuate shape.

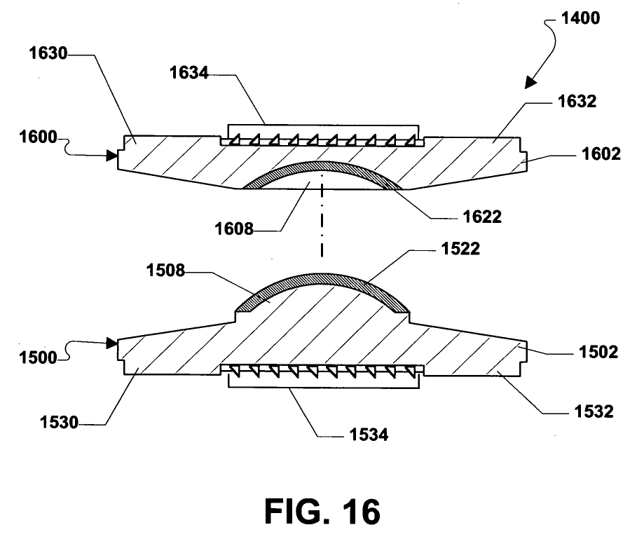

Referring to FIG. 16, the projection 1508 can include an inferior wear resistant layer 1522. In a particular embodiment, the inferior wear resistant layer 1522 can be formed by cross-linking the surface of the projection 1508. In a particular embodiment, depending on the type of material of which the projection 1508 is comprised, the surface of the projection 1508 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the projection 1508 can be cross-linked by exposing the surface of the projection 1508 to a cross-linking agent in the presence of a catalyst that promotes cross-linking in the subject material. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the projection 1508 can be cross-linked to a depth of about five millimeters (5 mm) or less, such as about three millimeters (3 mm) or less. In this manner, the material underlying the wear resistant layer 1522 can exhibit the typical material properties associated with the uncross-linked material that comprises the projection 1508.

Accordingly, the hardness of the wear resistant layer 1522 can be greater than the hardness of the underlying material. Further, the Young's modulus of the wear resistant layer 1522 can be greater than the Young's modulus of the underlying material. Also, the toughness of the wear resistant layer 1522 can be greater than the toughness of the underlying material.

Further, in a particular embodiment, the surface of the projection 1508 can be cross-linked in such a fashion that the hardness of the wear resistant layer 1522 decreases from a maximum at or near the surface of the wear resistant layer 1522 to the underlying uncross-linked material of the projection 1508. This can create a hardness gradient that substantially minimizes or eliminates an extreme change in hardness between the wear resistant layer 1522 and the projection 1508. Further, the hardness gradient substantially minimizes or eliminates the chance that the wear resistant layer 1522 may delaminate from the projection 1508.

In another particular embodiment, the underlying material of the projection 1508 may be cross-linked. However, in such a case, the mean or average cross-linking of the wear resistant layer 1522 may be greater than the underlying cross-linked material.

The cross-linking agent can be introduced or applied at various points during manufacture of the prosthetic disc in order to accommodate various manufacturing parameters, including the desired degree of cross-linking at or near the surface. Alternatively, the cross-linking agent can be introduced or applied post-manufacture, yet prior to implantation (e.g., by surgical staff or the like). Alternatively, in certain embodiments, the cross-linking agent can be introduced or applied after implantation. Further, a cross-linking agent can be introduced or applied at various points between the beginning of manufacture and the end of the implantation procedure. Two or more different cross-linking agents can be introduced or applied at various points, as desired, to obtain the proper degree of cross-linking in the desired location(s). The cross-linking agent(s) can be provided along with all or a portion of the prosthetic disc in kit form for ease of use in the field.

FIG. 14 through FIG. 18 and FIG. 20 also show that the inferior component 1500 can include a first inferior keel 1530, a second inferior keel 1532, and a plurality of inferior teeth 1534 that extend from the inferior bearing surface 1506. As shown, in a particular embodiment, the inferior keels 1530, 1532 and the inferior teeth 1534 are generally saw-tooth, or triangle, shaped. Further, the inferior keels 1530, 1532 and the inferior teeth 1534 are designed to engage cancellous bone, cortical bone, or a combination thereof of an inferior vertebra. Additionally, the inferior teeth 1534 can prevent the inferior component 1500 from moving with respect to an inferior vertebra after the intervertebral prosthetic disc 1400 is installed within the intervertebral space between the inferior vertebra and the superior vertebra.

In a particular embodiment, the inferior teeth 1534 can include other projections such as spikes, pins, blades, or a combination thereof that have any cross-sectional geometry.

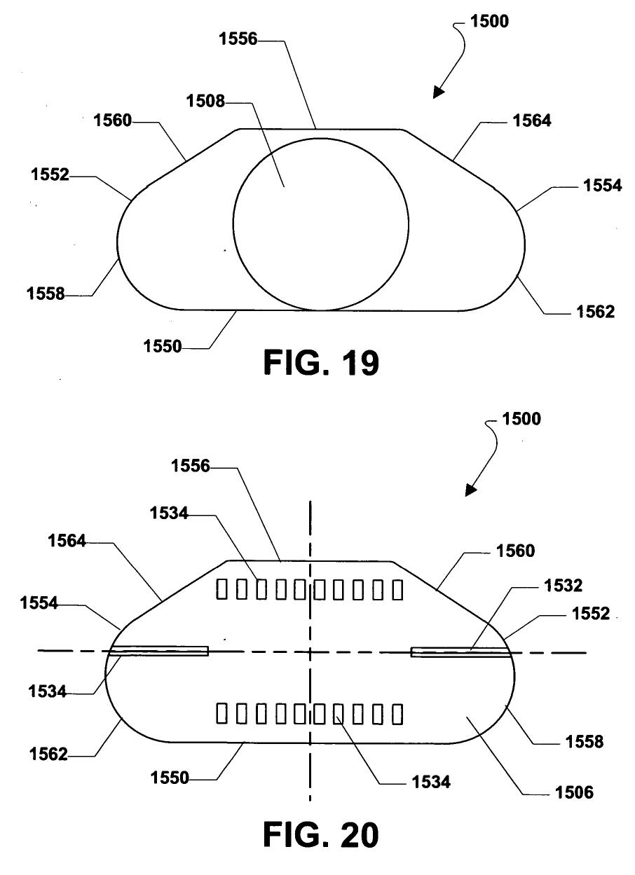

As illustrated in FIG. 19 and FIG. 20, the inferior component 1500 can be generally shaped to match the general shape of the vertebral body of a vertebra. For example, the inferior component 1500 can have a general trapezoid shape and the inferior component 1500 can include a posterior side 1550. A first lateral side 1552 and a second lateral side 1554 can extend from the posterior side 1550 to an anterior side 1556. In a particular embodiment, the first lateral side 1552 can include a curved portion 1558 and a straight portion 1560 that extends at an angle toward the anterior side 1556. Further, the second lateral side 1554 can also include a curved portion 1562 and a straight portion 1564 that extends at an angle toward the anterior side 1556.

As shown in FIG. 19 and FIG. 20, the anterior side 1556 of the inferior component 1500 can be relatively shorter than the posterior side 1550 of the inferior component 1500. Further, in a particular embodiment, the anterior side 1556 is substantially parallel to the posterior side 1550. As indicated in FIG. 19, the projection 1508 can be situated relative to the inferior articular surface 1504 such that the perimeter of the projection 1508 is tangential to the posterior side 1550 of the inferior component 1500. In alternative embodiments (not shown), the projection 1508 can be situated relative to the inferior articular surface 1504 such that the perimeter of the projection 1508 is tangential to the anterior side 1556 of the inferior component 1500 or tangential to both the anterior side 1556 and the posterior side 1550.

In a particular embodiment, the superior component 1600 can include a superior support plate 1602 that has a superior articular surface 1604 and a superior bearing surface 1606. In a particular embodiment, the superior articular surface 1604 can be generally rounded and the superior bearing surface 1606 can be generally flat.

As illustrated in FIG. 14 through FIG. 22, a depression 1608 extends into the superior articular surface 1604 of the superior support plate 1602. In a particular embodiment, the depression 1608 has a hemi-spherical shape. Alternatively, the depression 1608 can have an elliptical shape, a cylindrical shape, or other arcuate shape.

Referring to FIG. 16, the depression 1608 can a superior wear resistant layer 1622. In a particular embodiment, the superior wear resistant layer 1622 can be formed by cross-linking the surface of the depression 1608. In a particular embodiment, depending on the type of material of which the depression 1608 is comprised, the surface of the depression 1608 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the depression 1608 can be cross-linked by exposing the surface of the depression 1608 to a cross-linking agent in the presence of a catalyst that promotes cross-linking in the subject material. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol: 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the depression 1608 can be cross-linked to a depth of about five millimeters (5 mm) or less, such as about three millimeters (3 mm) or less. In this manner, the material underlying the wear resistant layer 1622 can exhibit the typical material properties associated with the uncross-linked material that comprises the depression 1608.

Accordingly, the hardness of the wear resistant layer 1622 can be greater than the hardness of the underlying material. Further, the Young's modulus of the wear resistant layer 1622 can be greater than the Young's modulus of the underlying material. Also, the toughness of the wear resistant layer 1622 can be greater than the toughness of the underlying material.

Further, in a particular embodiment, the surface of the depression 1608 can be cross-linked in such a fashion that the hardness of the wear resistant layer 1622 decreases from a maximum at or near the surface of the wear resistant layer 1622 to the underlying uncross-linked material of the depression 1608. This can create a hardness gradient that substantially minimizes or eliminates an extreme change in hardness between the wear resistant layer 1622 and the depression 1608. Further, the hardness gradient substantially minimizes or eliminates the chance that the wear resistant layer 1622 may delaminate from the depression 1608.

In another particular embodiment, the underlying material of the depression 1608 may be cross-linked. However, in such a case, the mean or average cross-linking of the wear resistant layer 1622 may be greater than the underlying cross-linked material.

FIG. 14 through FIG. 18 and FIG. 22 also show that the superior component 1600 can include a first superior keel 1630, a second superior keel 1632, and a plurality of superior teeth 1634 that extend from the superior bearing surface 1606. As shown, in a particular embodiment, the superior keels 1630, 1632 and the superior teeth 1634 are generally saw-tooth, or triangle, shaped. Further, the superior keels 1630, 1632 and the superior teeth 1634 are designed to engage cancellous bone, cortical bone, or a combination thereof, of a superior vertebra. Additionally, the superior teeth 1634 can prevent the superior component 1600 from moving with respect to a superior vertebra after the intervertebral prosthetic disc 1400 is installed within the intervertebral space between the inferior vertebra and the superior vertebra.

In a particular embodiment, the superior teeth 1634 can include other depressions such as spikes, pins, blades, or a combination thereof that have any cross-sectional geometry.

In a particular embodiment, the superior component 1600 can be shaped to match the shape of the inferior component 1500, shown in FIG. 19 and FIG. 20. Further, the superior component 1600 can be shaped to match the general shape of a vertebral body of a vertebra. For example, the superior component 1600 can have a general trapezoid shape and the superior component 1600 can include a posterior side 1650. A first lateral side 1652 and a second lateral side 1654 can extend from the posterior side 1650 to an anterior side 1656. In a particular embodiment, the first lateral side 1652 can include a curved portion 1658 and a straight portion 1660 that extends at an angle toward the anterior side 1656. Further, the second lateral side 1654 can also include a curved portion 1662 and a straight portion 1664 that extends at an angle toward the anterior side 1656.

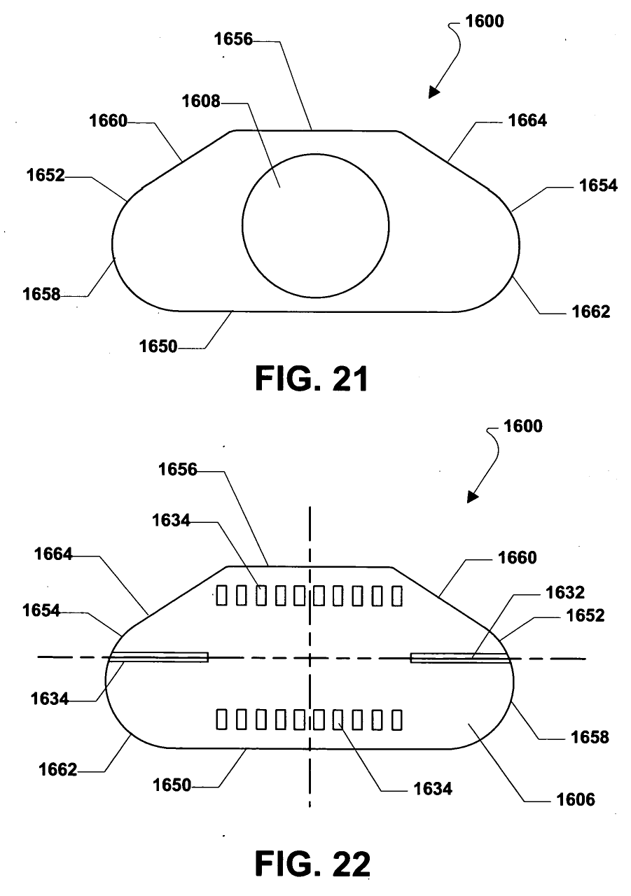

As shown in FIG. 21 and FIG. 22, the anterior side 1656 of the superior component 1600 can be relatively shorter than the posterior side 1650 of the superior component 1600. Further, in a particular embodiment, the anterior side 1656 is substantially parallel to the posterior side 1650.

In a particular embodiment, the overall height of the intervertebral prosthetic device 1400 can be in a range from six millimeters to twenty-two millimeters (6-22 mm). Further, the installed height of the intervertebral prosthetic device 1400 can be in a range from four millimeters to sixteen millimeters (4-16 mm). In a particular embodiment, the installed height can be substantially equivalent to the distance between an inferior vertebra and a superior vertebra when the intervertebral prosthetic device 1400 is installed there between.

In a particular embodiment, the length of the intervertebral prosthetic device 1400, e.g., along a longitudinal axis, can be in a range from thirty-three millimeters to fifty millimeters (33-50 mm). Additionally, the width of the intervertebral prosthetic device 1400, e.g., along a lateral axis, can be in a range from eighteen millimeters to twenty-nine millimeters (18-29 mm).

In a particular embodiment, the intervertebral prosthetic disc 1400 can be considered to be “low profile.” The low profile the intervertebral prosthetic device 1400 can allow the intervertebral prosthetic device 1400 to be implanted into an intervertebral space between an inferior vertebra and a superior vertebra laterally through a patient's psoas muscle, e.g., through an insertion device. Accordingly, the risk of damage to a patient's spinal cord or sympathetic chain can be substantially minimized. In alternative embodiments, all of the superior and inferior teeth 1518, 1618 can be oriented to engage in a direction substantially opposite the direction of insertion of the prosthetic disc into the intervertebral space.

Further, the intervertebral prosthetic disc 1400 can have a general “bullet” shape as shown in the posterior plan view, described herein. The bullet shape of the intervertebral prosthetic disc 1400 can further allow the intervertebral prosthetic disc 1400 to be inserted through the patient's psoas muscle while minimizing risk to the patient's spinal cord and sympathetic chain.

### DESCRIPTION OF A THIRD EMBODIMENT OF AN INTERVERTEBRAL PROSTHETIC DISC

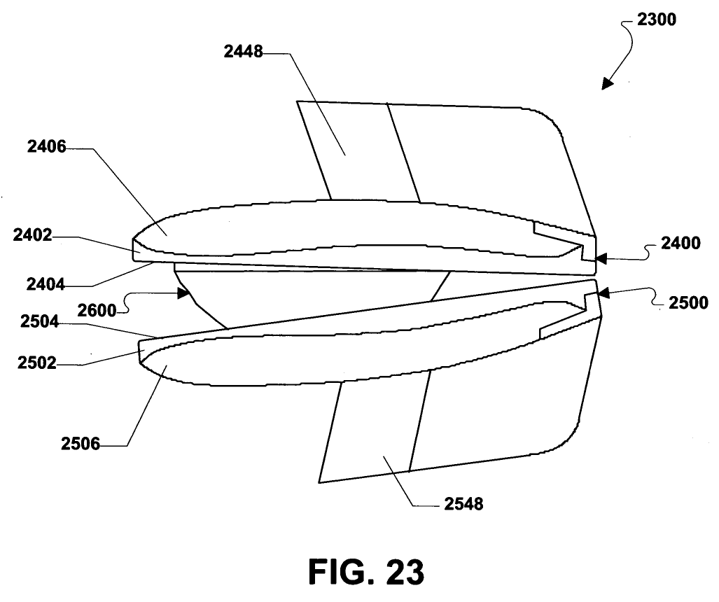

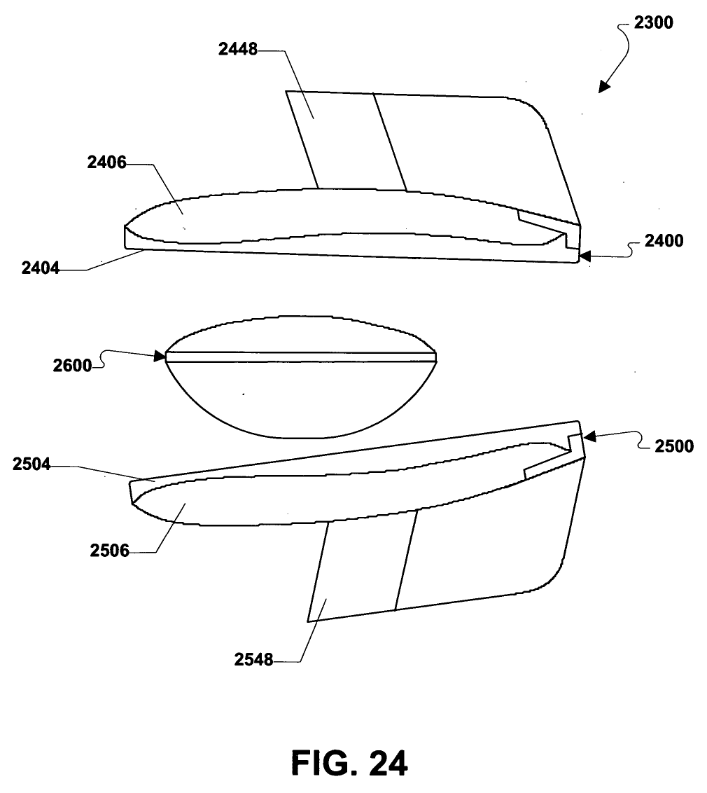

Referring to FIGS. 23 through 27 a third embodiment of an intervertebral prosthetic disc is shown and is generally designated 2300. As illustrated, the intervertebral prosthetic disc 2300 can include a superior component 2400, an inferior component 2500, and a nucleus 2600 disposed, or otherwise installed, there between. In a particular embodiment, the components 2400, 2500 and the nucleus 2600 can be made from one or more biocompatible materials. For example, the biocompatible materials can be one or more polymer materials.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, fluoropolyolefin, polybutadiene, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 2400, 2500 can be made from any other substantially rigid biocompatible materials.

In a particular embodiment, the superior component 2400 can include a superior support plate 2402 that has a superior articular surface 2404 and a superior bearing surface 2406. In a particular embodiment, the superior articular surface 2404 can be substantially flat and the superior bearing surface 2406 can be generally curved. In an alternative embodiment, at least a portion of the superior articular surface 2404 can be generally curved and the superior bearing surface 2406 can be substantially flat.

In a particular embodiment, after installation, the superior bearing surface 2406 can be in direct contact with vertebral bone, e.g., cortical bone and cancellous bone. Further, the superior bearing surface 2406 can be coated with a bone-growth promoting substance, e.g., a hydroxyapatite coating formed of calcium phosphate. Additionally, the superior bearing surface 2406 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

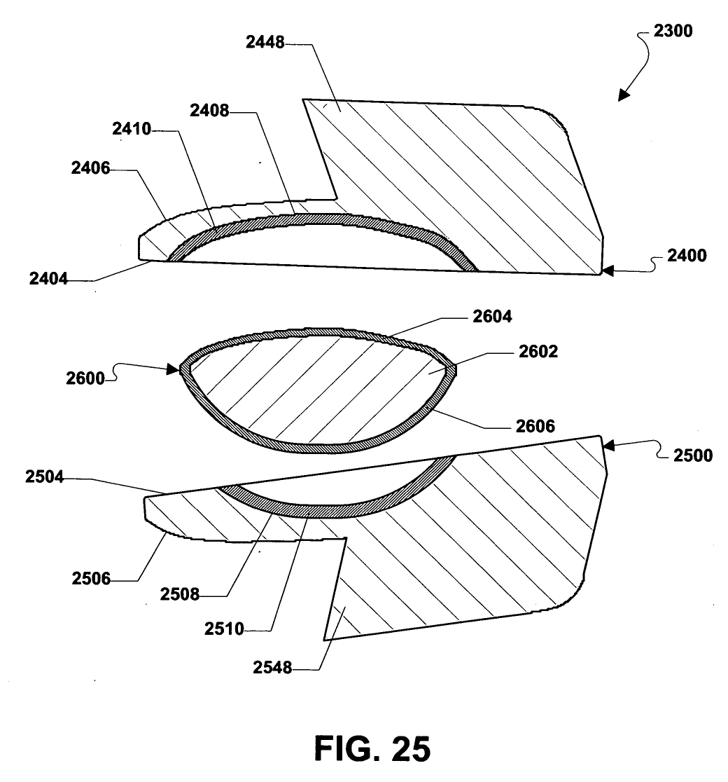

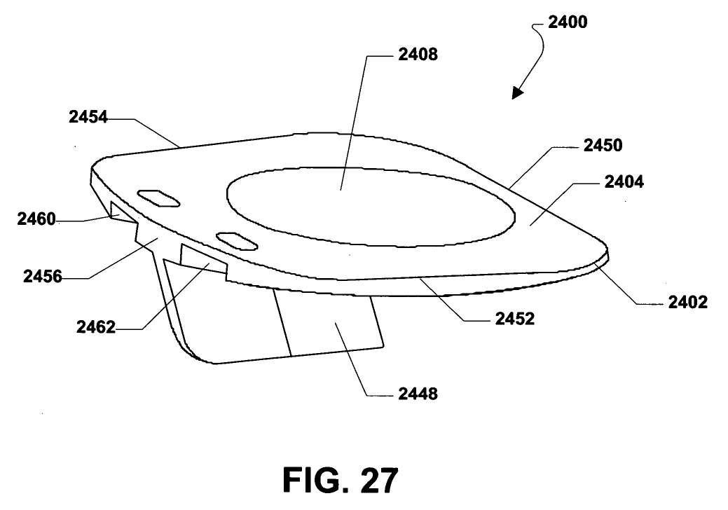

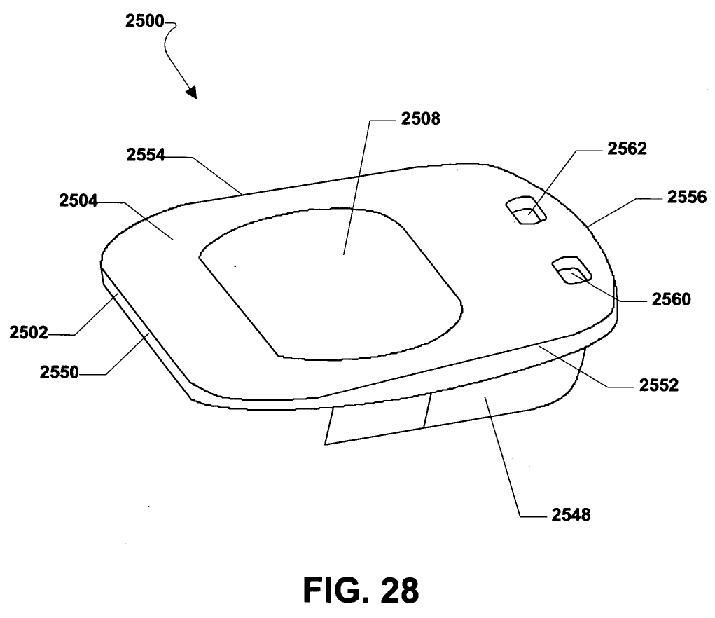

As illustrated in FIG. 25 and FIG. 27, a superior depression 2408 is established within the superior articular surface 2404 of the superior support plate 2402. In a particular embodiment, the superior depression 2408 has an arcuate shape. For example, the superior depression 2408 can have a hemispherical shape, an elliptical shape, a cylindrical shape, or any combination thereof.

FIG. 25 shows that superior depression 2408 can include a superior wear resistant layer 2410. In a particular embodiment, the superior wear resistant layer 2410 can be formed by cross-linking the surface of the superior depression 2408. In a particular embodiment, depending on the type of material of which the superior depression 2408 is comprised, the surface of the superior depression 2408 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the superior depression 2408 can be cross-linked by exposing the surface of the superior depression 2408 to a cross-linking agent in the presence of a catalyst that promotes cross-linking in the subject material. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the superior depression 2408 can be cross-linked to a depth of about five millimeters (5 mm) or less, such as about three millimeters (3 mm) or less. In this manner, the material underlying the wear resistant layer 2410 can exhibit the typical material properties associated with the uncross-linked material that comprises the superior depression 2408.

Accordingly, the hardness of the wear resistant layer 2410 can be greater than the hardness of the underlying material. Further, the Young's modulus of the wear resistant layer 2410 can be greater than the Young's modulus of the underlying material. Also, the toughness of the wear resistant layer 2410 can be greater than the toughness of the underlying material.

Further, in a particular embodiment, the surface of the superior depression 2408 can be cross-linked in such a fashion that the hardness of the wear resistant layer 2410 decreases from a maximum at or near the surface of the wear resistant layer 2410 to the underlying uncross-linked material of the superior depression 2408. This can create a hardness gradient that substantially minimizes or eliminates an extreme change in hardness between the wear resistant layer 2410 and the superior depression 2408. Further, the hardness gradient substantially minimizes or eliminates the chance that the wear resistant layer 2410 may delaminate from the superior depression 2408.

In another particular embodiment, the underlying material of the superior depression 2408 may be cross-linked. However, in such a case, the mean or average cross-linking of the wear resistant layer 2410 may be greater than the underlying cross-linked material.

The cross-linking agent can be introduced or applied at various points during manufacture of the prosthetic disc in order to accommodate various manufacturing parameters, including the desired degree of cross-linking at or near the surface. Alternatively, the cross-linking agent can be introduced or applied post-manufacture, yet prior to implantation (e.g., by surgical staff or the like). Alternatively, in certain embodiments, the cross-linking agent can be introduced or applied after implantation. Further, a cross-linking agent can be introduced or applied at various points between the beginning of manufacture and the end of the implantation procedure. Two or more different cross-linking agents can be introduced or applied at various points, as desired, to obtain the proper degree of cross-linking in the desired location(s). The cross-linking agent(s) can be provided along with all or a portion of the prosthetic disc in kit form for ease of use in the field.

FIG. 23 through FIG. 27 indicate that the superior component 2400 can include a superior keel 2448 that extends from superior bearing surface 2406. During installation, described below, the superior keel 2448 can at least partially engage a keel groove that can be established within a cortical rim of a superior vertebra. Further, the superior keel 2448 can be coated with a bone-growth promoting substance, e.g., a hydroxyapatite coating formed of calcium phosphate. In a particular embodiment, the superior keel 2448 does not include proteins, e.g., bone morphogenetic protein (BMP). Additionally, the superior keel 2448 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the superior component 2400, depicted in FIG. 27, can be generally rectangular in shape. For example, the superior component 2400 can have a substantially straight posterior side 2450. A first substantially straight lateral side 2452 and a second substantially straight lateral side 2454 can extend substantially perpendicularly from the posterior side 2450 to an anterior side 2456. In a particular embodiment, the anterior side 2456 can curve outward such that the superior component 2400 is wider through the middle than along the lateral sides 2452, 2454. Further, in a particular embodiment, the lateral sides 2452, 2454 are substantially the same length.

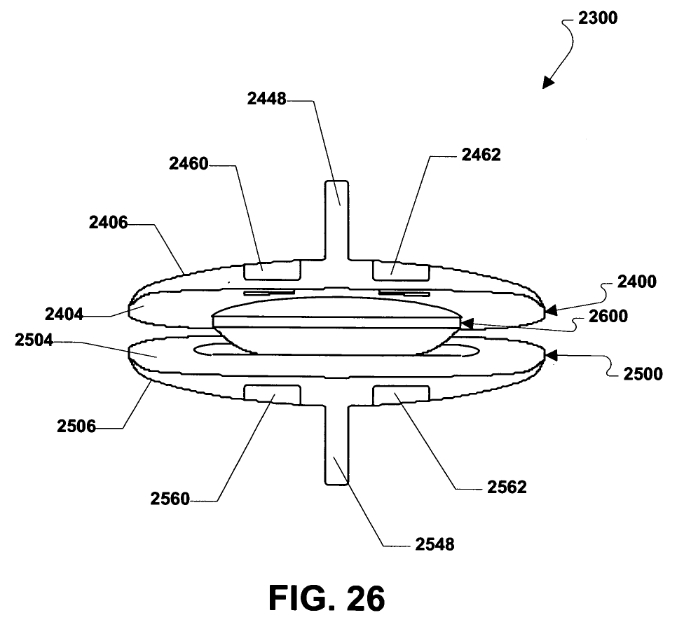

FIG. 26 shows that the superior component 2400 can include a first implant inserter engagement hole 2460 and a second implant inserter engagement hole 2462. In a particular embodiment, the implant inserter engagement holes 2460, 2462 are configured to receive a correspondingly shaped arm that extends from an implant inserter (not shown) that can be used to facilitate the proper installation of an intervertebral prosthetic disc, e.g., the intervertebral prosthetic disc 2300 shown in FIG. 23 through FIG. 27.

In a particular embodiment, the inferior component 2500 can include an inferior support plate 2502 that has an inferior articular surface 2504 and an inferior bearing surface 2506. In a particular embodiment, the inferior articular surface 2504 can be substantially flat and the inferior bearing surface 2506 can be generally curved. In an alternative embodiment, at least a portion of the inferior articular surface 2504 can be generally curved and the inferior bearing surface 2506 can be substantially flat.

In a particular embodiment, after installation, the inferior bearing surface 2506 can be in direct contact with vertebral bone, e.g., cortical bone and cancellous bone. Further, the inferior bearing surface 2506 can be coated with a bone-growth promoting substance, e.g., a hydroxyapatite coating formed of calcium phosphate. Additionally, the inferior bearing surface 2506 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 25 and FIG. 27, an inferior depression 2508 is established within the inferior articular surface 2504 of the inferior support plate 2502. In a particular embodiment, the inferior depression 2508 has an arcuate shape. For example, the inferior depression 2508 can have a hemispherical shape, an elliptical shape, a cylindrical shape, or any combination thereof.

FIG. 25 shows that the inferior depression 2508 can include an inferior wear resistant layer 2510. In a particular embodiment, the inferior wear resistant layer 2510 can be formed by cross-linking the surface of the inferior depression 2508. In a particular embodiment, depending on the type of material of which the inferior depression 2508 is comprised, the surface of the inferior depression 2508 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the inferior depression 2508 can be cross-linked by exposing the surface of the inferior depression 2508 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the inferior depression 2508 can be cross-linked to a depth of about five millimeters (5 mm) or less, such as about three millimeters (3 mm) or less. In this manner, the material underlying the wear resistant layer 2510 can exhibit the typical material properties associated with the uncross-linked material that comprises the inferior depression 2508.

Accordingly, the hardness of the wear resistant layer 2510 can be greater than the hardness of the underlying material. Further, the Young's modulus of the wear resistant layer 2510 can be greater than the Young's modulus of the underlying material. Also, the toughness of the wear resistant layer 2510 can be greater than the toughness of the underlying material.

Further, in a particular embodiment, the surface of the inferior depression 2508 can be cross-linked in such a fashion that the hardness of the wear resistant layer 2510 decreases from a maximum at or near the surface of the wear resistant layer 2510 to the underlying uncross-linked material of the inferior depression 2508. This can create a hardness gradient that substantially minimizes or eliminates an extreme change in hardness between the wear resistant layer 2510 and the inferior depression 2508. Further, the hardness gradient substantially minimizes or eliminates the chance that the wear resistant layer 2510 may delaminate from the inferior depression 2510.

In another particular embodiment, the underlying material of the inferior depression 2510 may be cross-linked. However, in such a case, the mean or average cross-linking of the wear resistant layer 2510 may be greater than the underlying cross-linked material.

FIG. 23 through FIG. 26 and FIG. 27 indicate that the inferior component 2500 can include an inferior keel 2548 that extends from inferior bearing surface 2506. During installation, described below, the inferior keel 2548 can at least partially engage a keel groove that can be established within a cortical rim of a vertebra. Further, the inferior keel 2548 can be coated with a bone-growth promoting substance, e.g., a hydroxyapatite coating formed of calcium phosphate. In a particular embodiment, the inferior keel 2548 does not include proteins, e.g., bone morphogenetic protein (BMP). Additionally, the inferior keel 2548 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

In a particular embodiment, the inferior component 2500, shown in FIG. 27, can be shaped to match the shape of the superior component 2400, shown in FIG. 27. Further, the inferior component 2500 can be generally rectangular in shape. For example, the inferior component 2500 can have a substantially straight posterior side 2550. A first substantially straight lateral side 2552 and a second substantially straight lateral side 2554 can extend substantially perpendicularly from the posterior side 2550 to an anterior side 2556. In a particular embodiment, the anterior side 2556 can curve outward such that the inferior component 2500 is wider through the middle than along the lateral sides 2552, 2554. Further, in a particular embodiment, the lateral sides 2552, 2554 are substantially the same length.

FIG. 26 shows that the inferior component 2500 can include a first implant inserter engagement hole 2560 and a second implant inserter engagement hole 2562. In a particular embodiment, the implant inserter engagement holes 2560, 2562 are configured to receive a correspondingly shaped arm that extends from an implant inserter (not shown) that can be used to facilitate the proper installation of an intervertebral prosthetic disc, e.g., the intervertebral prosthetic disc 2300 shown in FIG. 23 through FIG. 27.

FIG. 25 shows that the nucleus 2600 can include a core 2602. The core 2602 can include a superior wear resistant layer 2604 and an inferior resistant layer 2606. In a particular embodiment, the core 2602 can be a polymer material, e.g., one or more of the polymer materials described herein. Further, in a particular embodiment, the superior wear resistant layer 2604 and the inferior wear resistant layer 2606 can be established by cross-linking the surface of the core 2602.

In a particular embodiment, the superior wear resistant layer 2604 and the inferior resistant layer 2606 can be formed by cross-linking the surface of the core 2602. In a particular embodiment, depending on the type of material of which the core 2602 is comprised, the surface of the core 2602 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the core 2602 can be cross-linked by exposing the surface of the core 2602 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol, 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent or radiation source, the type of catalyst, etc. Also, in a particular embodiment, the surface of the core 2602 can be cross-linked to a depth of about five millimeters (5 mm) or less, such as about three millimeters (3 mm) or less. In this manner, the material underlying the wear resistant layers 2604, 2606 can exhibit the typical material properties associated with the uncross-linked material that comprises the core 2602.

Accordingly, the hardness of each wear resistant layer 2604, 2606 can be greater than the hardness of the underlying material. Further, the Young's modulus of each wear resistant layer 2604, 2606 can be greater than the Young's modulus of the underlying material. Also, the toughness of each wear resistant layer 2604, 2606 can be greater than the toughness of the underlying material.

Further, in a particular embodiment, the surface of the core 2602 can be cross-linked in such a fashion that the hardness of each wear resistant layer 2604, 2606 decreases from a maximum at or near the surface of each wear resistant layer 2604, 2606 to the underlying uncross-linked material of the core 2602. This can create a hardness gradient that substantially minimizes or eliminates an extreme change in hardness between each wear resistant layer 2604, 2606 and the core 2602. Further, the hardness gradient substantially minimizes or eliminates the chance that each wear resistant layer 2604, 2606 may delaminate from the core 2602.

In another particular embodiment, the underlying material of the core 2602 may be cross-linked. However, in such a case, the mean or average cross-linking of each wear resistant layer 2604, 2606 may be greater than the underlying cross-linked material of the core 2602.

In a particular embodiment, the superior wear resistant layer 2604 and the inferior wear resistant layer 2606 can each have an arcuate shape. For example, the superior wear resistant layer 2604 of the nucleus 2600 and the inferior wear resistant layer 2606 of the nucleus 2600 can have a hemispherical shape, an elliptical shape, a cylindrical shape, or any combination thereof. Further, in a particular embodiment, the superior wear resistant layer 2604 can be curved to match the superior depression 2408 of the superior component 2400. Also, in a particular embodiment, the inferior wear resistant layer 2606 of the nucleus 2600 can be curved to match the inferior depression 2508 of the inferior component 2500.

As shown in FIG. 23, the superior wear resistant layer 2604 of the nucleus 2600 can engage the superior wear resistant layer 2410 within the superior depression 2408 and can allow relative motion between the superior component 2400 and the nucleus 2600. Also, the inferior wear resistant layer 2606 of the nucleus 2600 can engage the inferior wear resistant layer 2510 within the inferior depression 2508 and can allow relative motion between the inferior component 2500 and the nucleus 2600. Accordingly, the nucleus 2600 can engage the superior component 2400 and the inferior component 2500 and the nucleus 2600 can allow the superior component 2400 to rotate with respect to the inferior component 2500.

In a particular embodiment, the overall height of the intervertebral prosthetic device 2300 can be in a range from fourteen millimeters to forty-six millimeters (14-46 mm). Further, the installed height of the intervertebral prosthetic device 2300 can be in a range from eight millimeters to sixteen millimeters (8-16 mm). In a particular embodiment, the installed height can be substantially equivalent to the distance between an inferior vertebra and a superior vertebra when the intervertebral prosthetic device 2300 is installed there between.

In a particular embodiment, the length of the intervertebral prosthetic device 2300, e.g., along a longitudinal axis, can be in a range from thirty millimeters to forty millimeters (30-40 mm). Additionally, the width of the intervertebral prosthetic device 2300, e.g., along a lateral axis, can be in a range from twenty-five millimeters to forty millimeters (25-40 mm).

### DESCRIPTION OF A FOURTH EMBODIMENT OF AN INTERVERTEBRAL PROSTHETIC DISC

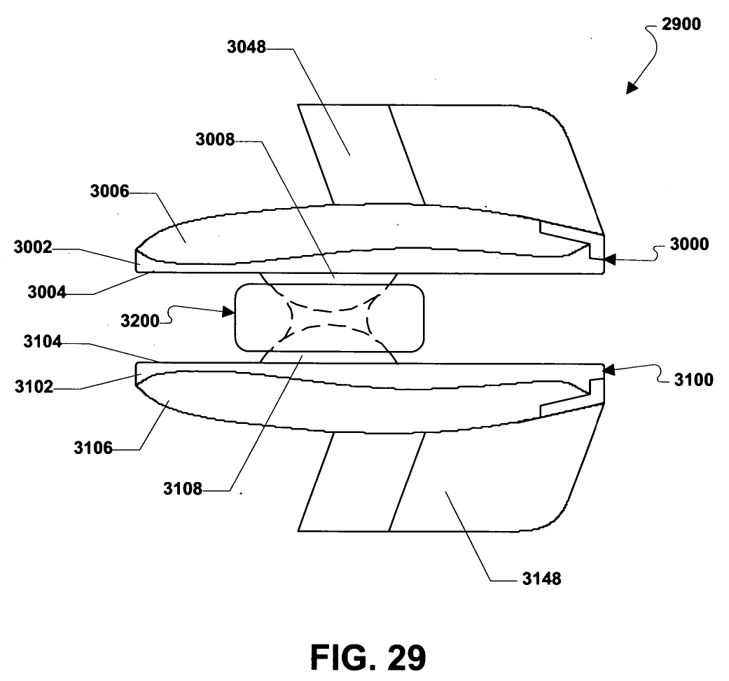

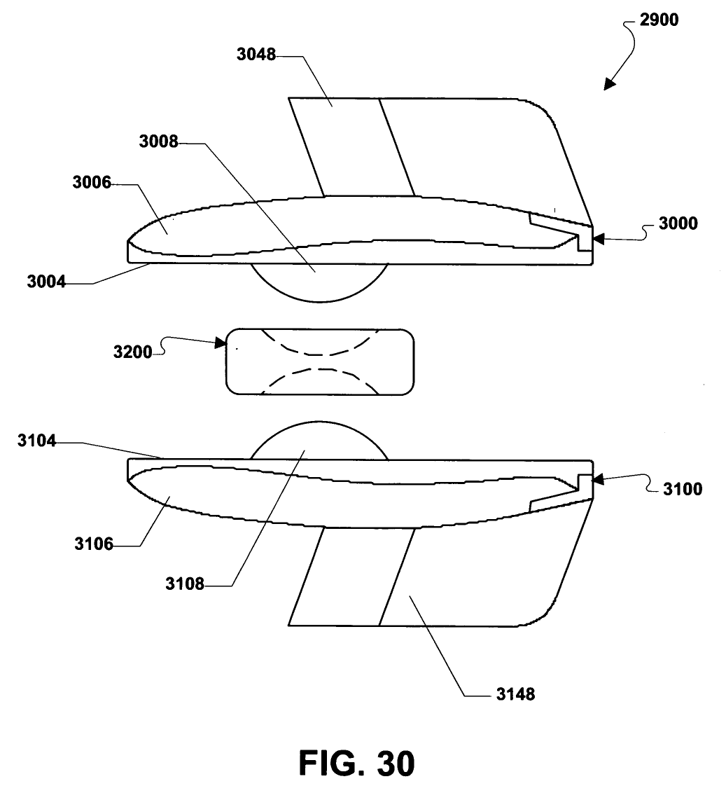

Referring to FIGS. 29 through 34, a fourth embodiment of an intervertebral prosthetic disc is shown and is generally designated 2900. As illustrated, the intervertebral prosthetic disc 2900 can include a superior component 3000, an inferior component 3100, and a nucleus 3200 disposed, or otherwise installed, there between. In a particular embodiment, the components 3000, 3100 and the nucleus 3200 can be made from one or more biocompatible materials. For example, the biocompatible materials can be one or more polymer materials.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, fluoropolyolefin, polybutadiene, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 3000, 3100 can be made from any other substantially rigid biocompatible materials.

In a particular embodiment, the superior component 3000 can include a superior support plate 3002 that has a superior articular surface 3004 and a superior bearing surface 3006. In a particular embodiment, the superior articular surface 3004 can be substantially flat and the superior bearing surface 3006 can be generally curved. In an alternative embodiment, at least a portion of the superior articular surface 3004 can be generally curved and the superior bearing surface 3006 can be substantially flat.

In a particular embodiment, after installation, the superior bearing surface 3006 can be in direct contact with vertebral bone, e.g., cortical bone and cancellous bone. Further, the superior bearing surface 3006 can be coated with a bone-growth promoting substance, e.g., a hydroxyapatite coating formed of calcium phosphate. Additionally, the superior bearing surface 3006 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 29 through FIG. 33, a superior projection 3008 extends from the superior articular surface 3004 of the superior support plate 3002. In a particular embodiment, the superior projection 3008 has an arcuate shape. For example, the superior depression 3008 can have a hemispherical shape, an elliptical shape, a cylindrical shape, or any combination thereof.

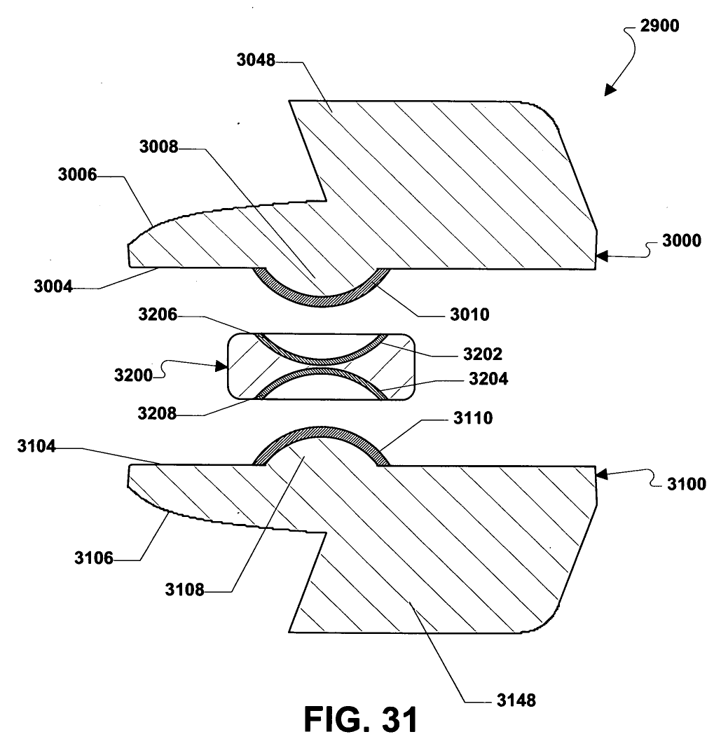

FIG. 31 shows that the superior projection 3008 can include a superior wear resistant layer 3010. In a particular embodiment, the superior wear resistant layer 3010 can be formed by cross-linking the surface of the superior projection 3008. In a particular embodiment, depending on the type of material of which the superior projection 3008 is comprised, the surface of the superior projection 3008 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the superior projection 3008 can be cross-linked by exposing the surface of the superior projection 3008 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof, 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the superior projection 3008 can be cross-linked to a depth of about five millimeters (5 mm) or less, such as about three millimeters (3 mm) or less. In this manner, the material underlying the wear resistant layer 3010 can exhibit the typical material properties associated with the uncross-linked material that comprises the superior projection 3008.

Accordingly, the hardness of the wear resistant layer 3010 can be greater than the hardness of the underlying material. Further, the Young's modulus of the wear resistant layer 3010 can be greater than the Young's modulus of the underlying material. Also, the toughness of the wear resistant layer 3010 can be greater than the toughness of the underlying material.

Further, in a particular embodiment, the surface of the superior projection 3008 can be cross-linked in such a fashion that the hardness of the wear resistant layer 3010 decreases from a maximum at or near the surface of the wear resistant layer 3010 to the underlying uncross-linked material of the superior projection 3008. This can create a hardness gradient that substantially minimizes or eliminates an extreme change in hardness between the wear resistant layer 3010 and the superior projection 3008. Further, the hardness gradient substantially minimizes or eliminates the chance that the wear resistant layer 3010 may delaminate from the superior projection 3008.

In another particular embodiment, the underlying material of the superior projection 3008 may be cross-linked. However, in such a case, the mean or average cross-linking of the wear resistant layer 3010 may be greater than the underlying cross-linked material.

The cross-linking agent can be introduced or applied at various points during manufacture of the prosthetic disc in order to accommodate various manufacturing parameters, including the desired degree of cross-linking at or near the surface. Alternatively, the cross-linking agent can be introduced or applied post-manufacture, yet prior to implantation (e.g., by surgical staff or the like). Alternatively, in certain embodiments, the cross-linking agent can be introduced or applied after implantation. Further, a cross-linking agent can be introduced or applied at various points between the beginning of manufacture and the end of the implantation procedure. Two or more different cross-linking agents can be introduced or applied at various points, as desired, to obtain the proper degree of cross-linking in the desired location(s). The cross-linking agent(s) can be provided along with all or a portion of the prosthetic disc in kit form for ease of use in the field.

FIG. 29 through FIG. 33 indicate that the superior component 3000 can include a superior keel 3048 that extends from superior bearing surface 3006. During installation, described below, the superior keel 3048 can at least partially engage a keel groove that can be established within a cortical rim of a superior vertebra. Further, the superior keel 3048 can be coated with a bone-growth promoting substance, e.g., a hydroxyapatite coating formed of calcium phosphate. In a particular embodiment, the superior keel 3048 does not include proteins, e.g., bone morphogenetic protein (BMP). Additionally, the superior keel 3048 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

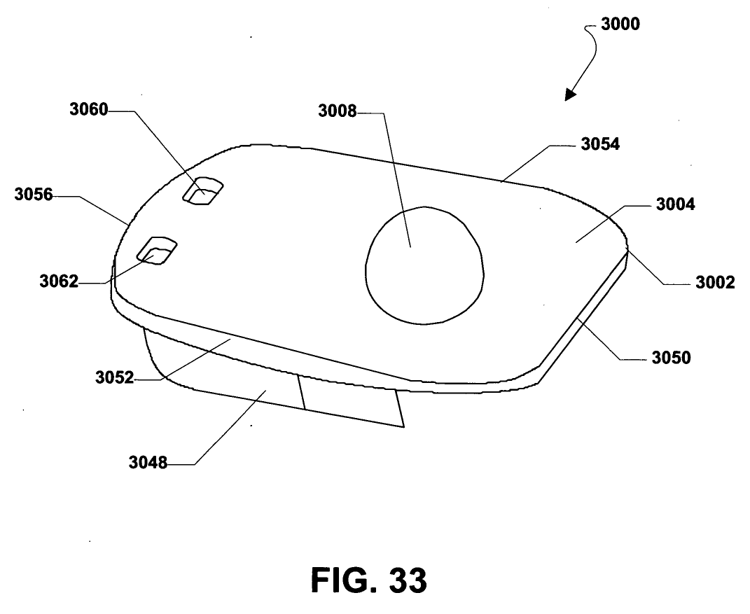

In a particular embodiment, the superior component 3000, depicted in FIG. 33, can be generally rectangular in shape. For example, the superior component 3000 can have a substantially straight posterior side 3050. A first substantially straight lateral side 3052 and a second substantially straight lateral side 3054 can extend substantially perpendicularly from the posterior side 3050 to an anterior side 3056. In a particular embodiment, the anterior side 3056 can curve outward such that the superior component 3000 is wider through the middle than along the lateral sides 3052, 3054. Further, in a particular embodiment, the lateral sides 3052, 3054 are substantially the same length.

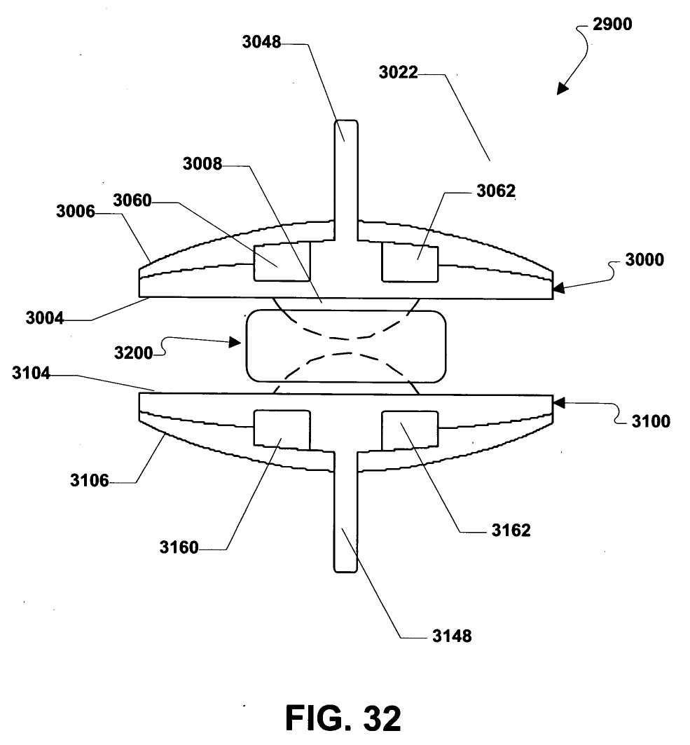

FIG. 32 shows that the superior component 3000 can include a first implant inserter engagement hole 3060 and a second implant inserter engagement hole 3062. In a particular embodiment, the implant inserter engagement holes 3060, 3062 are configured to receive a correspondingly shaped arm that extends from an implant inserter (not shown) that can be used to facilitate the proper installation of an intervertebral prosthetic disc, e.g., the intervertebral prosthetic disc 2200 shown in FIG. 29 through FIG. 34.

In a particular embodiment, the inferior component 3100 can include an inferior support plate 3102 that has an inferior articular surface 3104 and an inferior bearing surface 3106. In a particular embodiment, the inferior articular surface 3104 can be substantially flat and the inferior bearing surface 3106 can be generally curved. In an alternative embodiment, at least a portion of the inferior articular surface 3104 can be generally curved and the inferior bearing surface 3106 can be substantially flat.

In a particular embodiment, after installation, the inferior bearing surface 3106 can be in direct contact with vertebral bone, e.g., cortical bone and cancellous bone. Further, the inferior bearing surface 3106 can be coated with a bone-growth promoting substance, e.g., a hydroxyapatite coating formed of calcium phosphate. Additionally, the inferior bearing surface 3106 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

As illustrated in FIG. 29 through FIG. 32 and FIG. 34, an inferior projection 3108 can extend from the inferior articular surface 3104 of the inferior support plate 3102. In a particular embodiment, the inferior projection 3108 has an arcuate shape. For example, the inferior projection 3108 can have a hemispherical shape, an elliptical shape, a cylindrical shape, or any combination thereof.

FIG. 31 shows that the inferior projection 3108 can include an inferior wear resistant layer 3110. In a particular embodiment, the inferior wear resistant layer 3110 can be formed by cross-linking the surface of the inferior projection 3108. In a particular embodiment, depending on the type of material of which the inferior projection 3108 is comprised, the surface of the inferior projection 3108 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the inferior projection 3108 can be cross-linked by exposing the surface of the inferior projection 3108 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the inferior projection 3108 can be cross-linked to a depth of about five millimeters (5 mm) or less, such as about three millimeters (3 mm) or less. In this manner, the material underlying the wear resistant layer 3110 can exhibit the typical material properties associated with the uncross-linked material that comprises the inferior projection 3108.

Accordingly, the hardness of the wear resistant layer 3110 can be greater than the hardness of the underlying material. Further, the Young's modulus of the wear resistant layer 3110 can be greater than the Young's modulus of the underlying material. Also, the toughness of the wear resistant layer 3110 can be greater than the toughness of the underlying material.

Further, in a particular embodiment, the surface of the inferior projection 3108 can be cross-linked in such a fashion that the hardness of the wear resistant layer 3110 decreases from a maximum at or near the surface of the wear resistant layer 3110 to the underlying uncross-linked material of the inferior projection 3108. This can create a hardness gradient that substantially minimizes or eliminates an extreme change in hardness between the wear resistant layer 3110 and the inferior projection 3108. Further, the hardness gradient substantially minimizes or eliminates the chance that the wear resistant layer 3110 may delaminate from the inferior projection 3108.

In another particular embodiment, the underlying material of the inferior projection 3108 may be cross-linked. However, in such a case, the mean or average cross-linking of the wear resistant layer 3110 may be greater than the underlying cross-linked material.

FIG. 29 through FIG. 32 and FIG. 34 indicate that the inferior component 3100 can include an inferior keel 3148 that extends from inferior bearing surface 3106. During installation, described below, the inferior keel 3148 can at least partially engage a keel groove that can be established within a cortical rim of a vertebra. Further, the inferior keel 3148 can be coated with a bone-growth promoting substance, e.g., a hydroxyapatite coating formed of calcium phosphate. In a particular embodiment, the inferior keel 3148 does not include proteins, e.g., bone morphogenetic protein (BMP). Additionally, the inferior keel 3148 can be roughened prior to being coated with the bone-growth promoting substance to further enhance bone on-growth or in-growth. In a particular embodiment, the roughening process can include acid etching; knurling; application of a bead coating (porous or non-porous), e.g., cobalt chrome beads; application of a roughening spray, e.g., titanium plasma spray (TPS); laser blasting; or any other similar process or method.

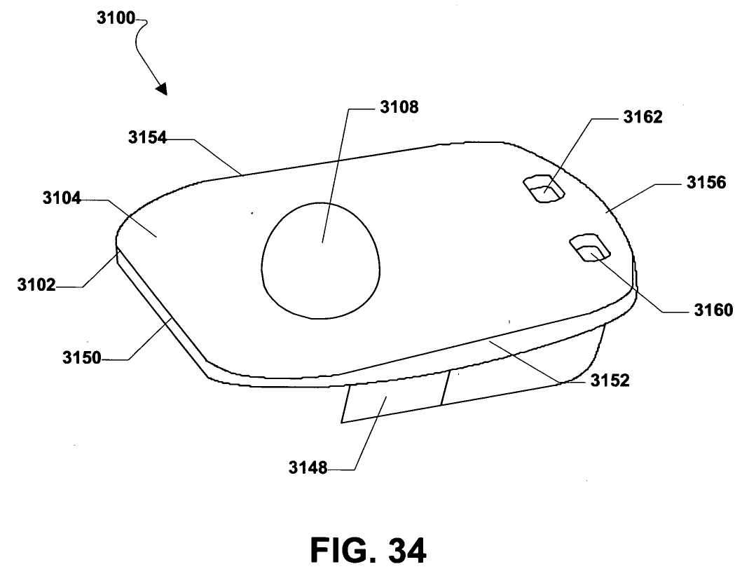

In a particular embodiment, the inferior component 3100, shown in FIG. 34, can be shaped to match the shape of the superior component 3000, shown in FIG. 33. Further, the inferior component 3100 can be generally rectangular in shape. For example, the inferior component 3100 can have a substantially straight posterior side 3150. A first substantially straight lateral side 3152 and a second substantially straight lateral side 3154 can extend substantially perpendicularly from the posterior side 3150 to an anterior side 3156. In a particular embodiment, the anterior side 3156 can curve outward such that the inferior component 3100 is wider through the middle than along the lateral sides 3152, 3154. Further, in a particular embodiment, the lateral sides 3152, 3154 are substantially the same length.

FIG. 32 shows that the inferior component 3100 can include a first implant inserter engagement hole 3160 and a second implant inserter engagement hole 3162. In a particular embodiment, the implant inserter engagement holes 3160, 3162 are configured to receive a correspondingly shaped arm that extends from an implant inserter (not shown) that can be used to facilitate the proper installation of an intervertebral prosthetic disc, e.g., the intervertebral prosthetic disc 2200 shown in FIG. 29 through FIG. 34.

FIG. 31 shows that the nucleus 3200 can include a superior depression 3202 and an inferior depression 3204. In a particular embodiment, the superior depression 3202 and the inferior depression 3204 can each have an arcuate shape. For example, the superior depression 3202 of the nucleus 3200 and the inferior depression 3204 of the nucleus 3200 can have a hemispherical shape, an elliptical shape, a cylindrical shape, or any combination thereof. Further, in a particular embodiment, the superior depression 3202 can be curved to match the superior projection 3008 of the superior component 3000. Also, in a particular embodiment, the inferior depression 3204 of the nucleus 3200 can be curved to match the inferior projection 3108 of the inferior component 3100.

FIG. 31 shows that the superior depression 3202 of the nucleus 3200 can include a superior wear resistant layer 3206. Also, the inferior depression 3204 of the nucleus 3200 can include an inferior wear resistant layer 3208. In a particular embodiment, the superior wear resistant layer 3206 and the inferior wear resistant layer 3208 can be formed by cross-linking the surface of the superior depression 3202 and by cross-linking the surface of the inferior depression 3204, respectively.

In a particular embodiment, depending on the type of material of which the depressions 3202, 3204 are comprised, the surface of each depression 3202, 3204 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of each depression 3202, 3204 can be cross-linked by exposing the surface of each depression 3202, 3204 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of each depression 3202, 3204 can be cross-linked to a depth of about five millimeters (5 mm) or less, such as about three millimeters (3 mm) or less. In this manner, the material underlying each wear resistant layer 3206, 3208 can exhibit the typical material properties associated with the uncross-linked material that comprises the depressions 3202, 3204.

Accordingly, the hardness of each wear resistant layer 3206, 3208 can be greater than the hardness of the underlying material. Further, the Young's modulus of each wear resistant layer 3206, 3208 can be greater than the Young's modulus of the underlying material. Also, the toughness of each wear resistant layer 3206, 3208 can be greater than the toughness of the underlying material.

Further, in a particular embodiment, the surface of each depression 3202, 3204 can be cross-linked in such a fashion that the hardness of each wear resistant layer 3206, 3208 decreases from a maximum at or near the surface of each wear resistant layer 3206, 3208 to the underlying uncross-linked material of the depressions 3202, 3204. This can create a hardness gradient that substantially minimizes or eliminates an extreme change in hardness between each wear resistant layer 3206, 3208 and the respective depression 3202, 3204. Further, the hardness gradient substantially minimizes or eliminates the chance that each wear resistant layer 3206, 3208 may delaminate from the respective depression 3202, 3204.

In another particular embodiment, the underlying material of the depressions 3202, 3204 may be cross-linked. However, in such a case, the mean or average cross-linking of the each wear resistant layer 3206, 3208 may be greater than the underlying cross-linked material.

As shown in FIG. 29, the superior wear resistant layer 3206 of the nucleus 3200 can engage the superior wear resistant layer 3010 of the superior component 3000 and can allow relative motion between the superior component 3000 and the nucleus 3200. Also, the inferior wear resistant layer 3208 of the nucleus 3200 can engage the inferior wear resistant layer 3110 of the inferior component 3100 and can allow relative motion between the inferior component 3100 and the nucleus 3200. Accordingly, the nucleus 3200 can engage the superior component 3000 and the inferior component 3100, and the nucleus 3200 can allow the superior component 3000 to rotate with respect to the inferior component 3100.

In a particular embodiment, the overall height of the intervertebral prosthetic device 2900 can be in a range from fourteen millimeters to forty-six millimeters (14-46 mm). Further, the installed height of the intervertebral prosthetic device 2900 can be in a range from eight millimeters to sixteen millimeters (8-16 mm). In a particular embodiment, the installed height can be substantially equivalent to the distance between an inferior vertebra and a superior vertebra when the intervertebral prosthetic device 2900 is installed there between.

In a particular embodiment, the length of the intervertebral prosthetic device 2900, e.g., along a longitudinal axis, can be in a range from thirty millimeters to forty millimeters (30-40 mm). Additionally, the width of the intervertebral prosthetic device 2900, e.g., along a lateral axis, can be in a range from twenty-five millimeters to forty millimeters (25-40 mm).

### DESCRIPTION OF A FIFTH EMBODIMENT OF AN INTERVERTEBRAL PROSTHETIC DISC

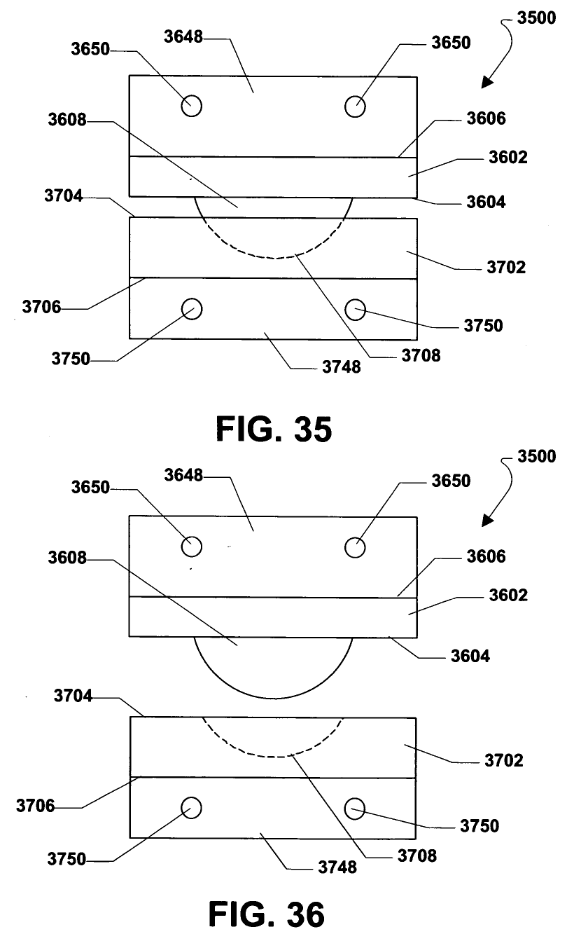

Referring to FIGS. 35 through 39 a fifth embodiment of an intervertebral prosthetic disc is shown and is generally designated 3500. As illustrated, the intervertebral prosthetic disc 3500 can include a superior component 3600 and an inferior component 3700. In a particular embodiment, the components 3600, 3700 can be made from one or more biocompatible materials. For example, the biocompatible materials can be one or more polymer materials.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, fluoropolyolefin, polybutadiene, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 3600, 3700 can be made from any other substantially rigid biocompatible materials.

In a particular embodiment, the superior component 3600 can include a superior support plate 3602 that has a superior articular surface 3604 and a superior bearing surface 3606. In a particular embodiment, the superior articular surface 3604 can be substantially flat and the superior bearing surface 3606 can be substantially flat. In an alternative embodiment, at least a portion of the superior articular surface 3604 can be generally curved and at least a portion of the superior bearing surface 3606 can be generally curved.

As illustrated in FIG. 35 through FIG. 37, a projection 3608 extends from the superior articular surface 3604 of the superior support plate 3602. In a particular embodiment, the projection 3608 has a hemi-spherical shape. Alternatively, the projection 3608 can have an elliptical shape, a cylindrical shape, or other arcuate shape.

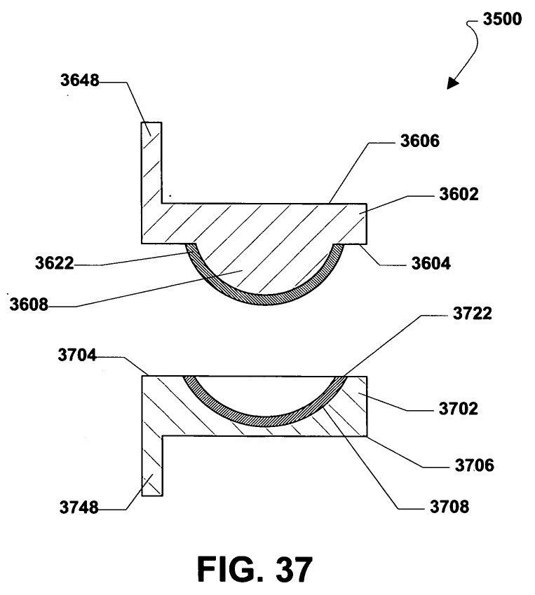

Referring to FIG. 37, the projection 3608 can include a superior wear resistant layer 3622. In a particular embodiment, the superior wear resistant layer 3622 can be formed by cross-linking the surface of the projection 3608. In a particular embodiment, depending on the type of material of which the projection 3608 is comprised, the surface of the projection 3608 can be cross-linked using a cross-linking agent. The cross-linking agent can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the projection 3608 can be cross-linked by exposing the surface of the projection 3608 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof. In a particular embodiment, the amount of cross-linking can vary depending on the type of material to be cross-linked, the time of exposure of the material to the cross-linking agent, the type of catalyst, etc. Also, in a particular embodiment, the surface of the projection 3608 can be cross-linked to a depth of about five millimeters (5 mm) or less, such as about three millimeters (3 mm) or less. In this manner, the material underlying the wear resistant layer 3622 can exhibit the typical material properties associated with the uncross-linked material that comprises the projection 3608.

Accordingly, the hardness of the wear resistant layer 3622 can be greater than the hardness of the underlying material. Further, the Young's modulus of the wear resistant layer 3622 can be greater than the Young's modulus of the underlying material. Also, the toughness of the wear resistant layer 3622 can be greater than the toughness of the underlying material.

Further, in a particular embodiment, the surface of the projection 3608 can be cross-linked in such a fashion that the hardness of the wear resistant layer 3622 decreases from a maximum at or near the surface of the wear resistant layer 3622 to the underlying uncross-linked material of the projection 3608. This can create a hardness gradient that substantially minimizes or eliminates an extreme change in hardness between the wear resistant layer 3622 and the projection 3608. Further, the hardness gradient substantially minimizes or eliminates the chance that the wear resistant layer 3622 may delaminate from the projection.

In another particular embodiment, the underlying material of the projection 3608 may be cross-linked. However, in such a case, the mean or average cross-linking of the wear resistant layer 3622 may be greater than the underlying cross-linked material.

The cross-linking agent can be introduced or applied at various points during manufacture of the prosthetic disc in order to accommodate various manufacturing parameters, including the desired degree of cross-linking at or near the surface. Alternatively, the cross-linking agent can be introduced or applied post-manufacture, yet prior to implantation (e.g., by surgical staff or the like). Alternatively, in certain embodiments, the cross-linking agent can be introduced or applied after implantation. Further, a cross-linking agent can be introduced or applied at various points between the beginning of manufacture and the end of the implantation procedure. Two or more different cross-linking agents can be introduced or applied at various points, as desired, to obtain the proper degree of cross-linking in the desired location(s). The cross-linking agent(s) can be provided along with all or a portion of the prosthetic disc in kit form for ease of use in the field.

FIG. 35 through FIG. 37 also show that the superior component 3600 can include a superior bracket 3648 that can extend substantially perpendicular from the superior support plate 3602. Further, the superior bracket 3648 can include at least one hole 3650. In a particular embodiment, a fastener, e.g., a screw, can be inserted through the hole 3650 in the superior bracket 3648 in order to attach, or otherwise affix, the superior component 3600 to a superior vertebra.

The superior bearing surface 3606 can be coated with a bone-growth promoting substance, e.g., a hydroxyapatite coating formed of calcium phosphate. Additionally, the superior bearing surface 3606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 38, the superior component 3600 can be generally rectangular in shape. For example, the superior component 3600 can have a substantially straight posterior side 3660. A first straight lateral side 3662 and a second substantially straight lateral side 3664 can extend substantially perpendicular from the posterior side 3660 to a substantially straight anterior side 3666. In a particular embodiment, the anterior side 3666 and the posterior side 3660 are substantially the same length. Further, in a particular embodiment, the lateral sides 3662, 3664 are substantially the same length.

In a particular embodiment, the inferior component 3700 can include an inferior support plate 3702 that has an inferior articular surface 3704 and an inferior bearing surface 3706. In a particular embodiment, the inferior articular surface 3704 can be generally curved and the inferior bearing surface 3706 can be substantially flat. In an alternative embodiment, the inferior articular surface 3704 can be substantially flat and at least a portion of the inferior bearing surface 3706 can be generally curved.

As illustrated in FIG. 35 through FIG. 37, a depression 3708 extends into the inferior articular surface 3704 of the inferior support plate 3702. In a particular embodiment, the depression 3708 is sized and shaped to receive the projection 3608 of the superior component 3600. For example, the depression 3708 can have a hemi-spherical shape. Alternatively, the depression 3708 can have an elliptical shape, a cylindrical shape, or other arcuate shape.

Referring to FIG. 37, the depression 3708 can include an inferior wear resistant layer 3722. In a particular embodiment, the inferior wear resistant layer 3722 can be formed by cross-linking the surface of the depression 3708. In a particular embodiment, depending on the type of material of which the depression 3708 is comprised, the surface of the depression 3708 can be cross-linked using a cross-linking agent. Acceptable cross-linking agents can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the depression 3708 can be cross-linked by exposing the surface of the depression 3708 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the depression 3708 can be cross-linked to a depth of about five millimeters (5 mm) or less, such as about three millimeters (3 mm) or less. In this manner, the material underlying the wear resistant layer 3722 can exhibit the typical material properties associated with the uncross-linked material that comprises the depression 3708.

Accordingly, the hardness of the wear resistant layer 3722 can be greater than the hardness of the underlying material. Further, the Young's modulus of the wear resistant layer 3722 can be greater than the Young's modulus of the underlying material. Also, the toughness of the wear resistant layer 3722 can be greater than the toughness of the underlying material.

Further, in a particular embodiment, the surface of the depression 3708 can be cross-linked in such a fashion that the hardness of the wear resistant layer 3722 decreases from a maximum at or near the surface of the wear resistant layer 3722 to the underlying uncross-linked material of the depression 3708. This can create a hardness gradient that substantially minimizes or eliminates an extreme change in hardness between the wear resistant layer 3722 and the depression 3708. Further, the hardness gradient substantially minimizes or eliminates the chance that the wear resistant layer 3722 may delaminate from the depression 3708.

In another particular embodiment, the underlying material of the depression 3708 may be cross-linked. However, in such a case, the mean or average cross-linking of the wear resistant layer 3722 may be greater than the underlying cross-linked material.

FIG. 35 through FIG. 37 also show that the inferior component 3700 can include an inferior bracket 3748 that can extend substantially perpendicular from the inferior support plate 3702. Further, the inferior bracket 3748 can include a hole 3750. In a particular embodiment, a fastener, e.g., a screw, can be inserted through the hole 3750 in the inferior bracket 3748 in order to attach, or otherwise affix, the inferior component 3700 to an inferior vertebra.

The inferior bearing surface 3706 can be coated with a bone-growth promoting substance, e.g., a hydroxyapatite coating formed of calcium phosphate. Additionally, the inferior bearing surface 3706 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

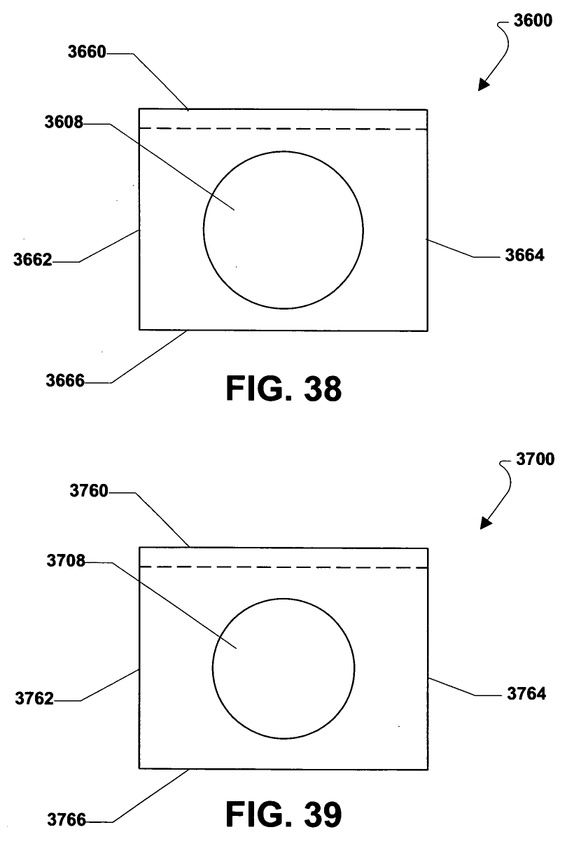

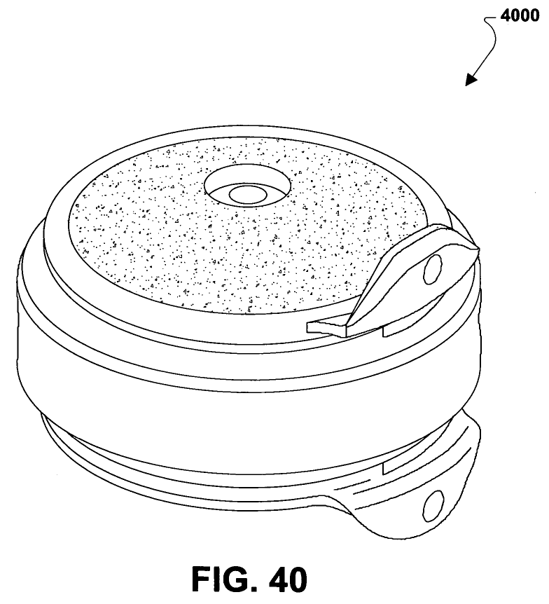

As illustrated in FIG. 39, the inferior component 3700 can be generally rectangular in shape. For example, the inferior component 3700 can have a substantially straight posterior side 3760. A first straight lateral side 3762 and a second substantially straight lateral side 3764 can extend substantially perpendicular from the posterior side 3760 to a substantially straight anterior side 3766. In a particular embodiment, the anterior side 3766 and the posterior side 3760 are substantially the same length. Further, in a particular embodiment, the lateral sides 3762, 3764 are substantially the same length.

In a particular embodiment, the overall height of the intervertebral prosthetic device 3500 can be in a range from fourteen millimeters to forty-six millimeters (14-46 mm). Further, the installed height of the intervertebral prosthetic device 3500 can be in a range from eight millimeters to sixteen millimeters (8-16 mm). In a particular embodiment, the installed height can be substantially equivalent to the distance between an inferior vertebra and a superior vertebra when the intervertebral prosthetic device 3500 is installed there between.

In a particular embodiment, the length of the intervertebral prosthetic device 3500, e.g., along a longitudinal axis, can be in a range from thirty millimeters to forty millimeters (30-40 mm). Additionally, the width of the intervertebral prosthetic device 3500, e.g., along a lateral axis, can be in a range from twenty-five millimeters to forty millimeters (25-40 mm). Moreover, in a particular embodiment, each bracket 3648, 3748 can have a height in a range from three millimeters to fifteen millimeters (3-15 mm).

### DESCRIPTION OF A SIXTH EMBODIMENT OF AN INTERVERTEBRAL PROSTHETIC DISC

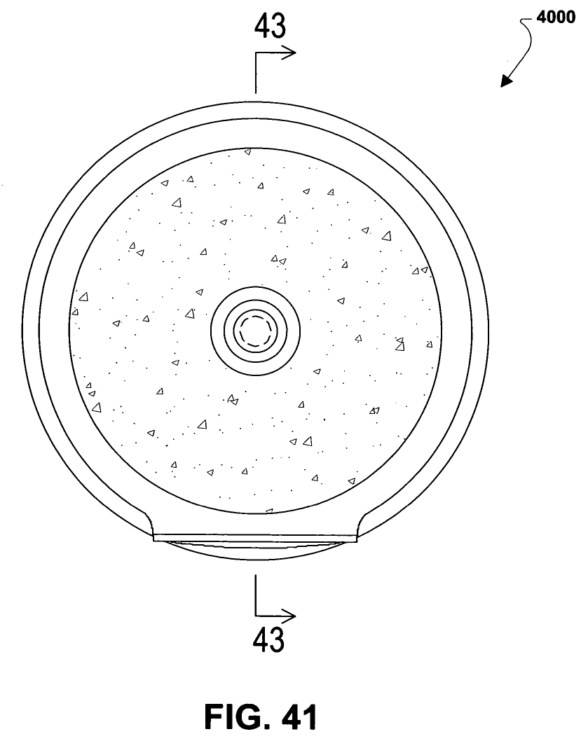

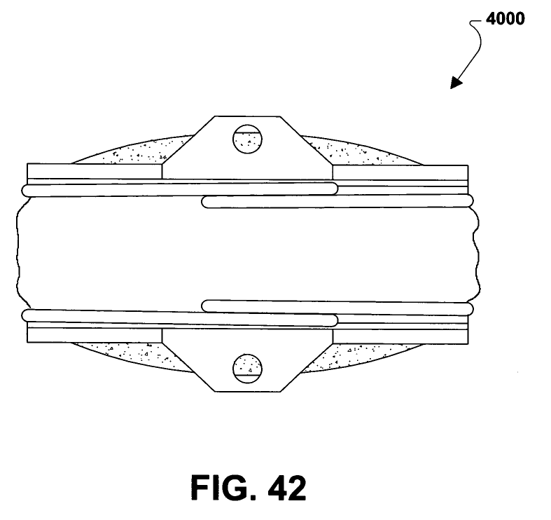

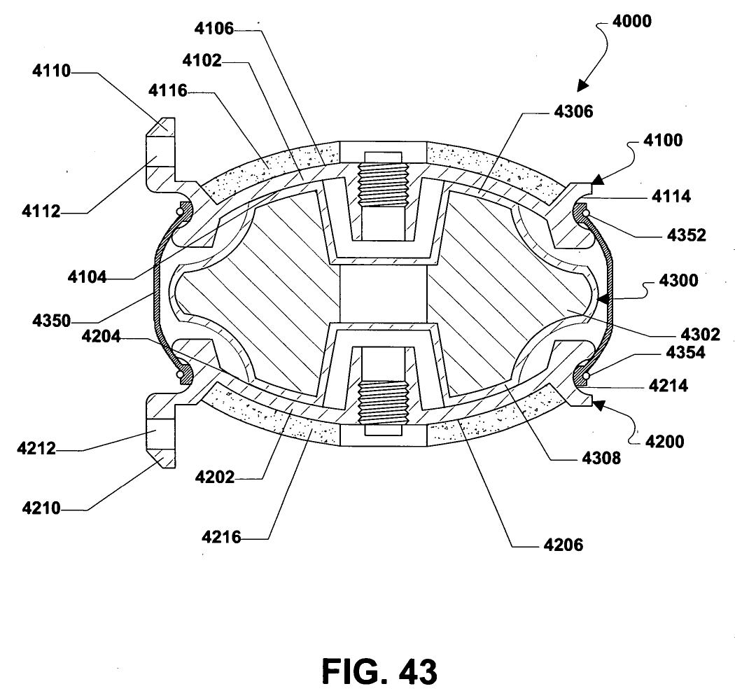

Referring to FIGS. 40 through 43, a sixth embodiment of an intervertebral prosthetic disc is shown and is generally designated 4000. As illustrated in FIG. 43, the intervertebral prosthetic disc 4000 can include a superior component 4100, an inferior component 4200, and a nucleus 4300 disposed, or otherwise installed, there between. In a particular embodiment, a sheath 4350 surrounds the nucleus 4300 and is affixed or otherwise coupled to the superior component 4100 and the inferior component 4200. In a particular embodiment, the components 4100, 4200 and the nucleus 4300 can be made from one or more biocompatible materials. For example, the biocompatible materials can be one or more polymer materials.

The polymer materials can include polyurethane materials, polyolefin materials, polyaryletherketone (PAEK) materials, silicone materials, hydrogel materials, or a combination thereof. Further, the polyolefin materials can include polypropylene, polyethylene, halogenated polyolefin, fluoropolyolefin, polybutadiene, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. Alternatively, the components 4100, 4200 can be made from any other substantially rigid biocompatible materials.

In a particular embodiment, the superior component 4100 can include a superior support plate 4102 that has a superior articular surface 4104 and a superior bearing surface 4106. In a particular embodiment, the superior support plate 4102 can be generally rounded, generally cup shaped, or generally bowl shaped. Further, in a particular embodiment, the superior articular surface 4104 can be generally rounded or generally curved and the superior bearing surface 4106 can be generally rounded or generally curved.

FIG. 43 also shows that the superior support plate 4102 can include a superior bracket 4110 that can extend substantially perpendicular from the superior support plate 4102. The superior bracket 4110 can include a hole 4112. In a particular embodiment, a fastener, e.g., a screw, can be inserted through the hole 4112 in the superior bracket 4110 in order to attach, or otherwise affix, the superior component 4100 to a superior vertebra.

Moreover, the superior support plate 4102 includes a superior channel 4114 established around the perimeter of the superior support plate 4102. In a particular embodiment, a portion of the sheath 4300 can be held within the superior channel 4114 using a superior retaining ring 4352.

As depicted in FIG. 43, the superior support plate 4102 can include a bone growth promoting layer 4116 disposed, or otherwise deposited, on the superior bearing surface 4106. In a particular embodiment, the bone growth promoting layer 4116 can include a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

In a particular embodiment, the inferior component 4200 can include an inferior support plate 4202 that has an inferior articular surface 4204 and an inferior bearing surface 4206. In a particular embodiment, the inferior support plate 4202 can be generally rounded, generally cup shaped, or generally bowl shaped. Further, in a particular embodiment, the inferior articular surface 4204 can be generally rounded or generally curved and the inferior bearing surface 4206 can be generally rounded or generally curved.

FIG. 43 also shows that the inferior support plate 4202 can include an inferior bracket 4210 that can extend substantially perpendicular from the inferior support plate 4202. The inferior bracket 4210 can include a hole 4212. In a particular embodiment, a fastener, e.g., a screw, can be inserted through the hole 4212 in the inferior bracket 4210 in order to attach, or otherwise affix, the inferior component 4200 to an inferior vertebra.

Moreover, the inferior support plate 4202 includes an inferior channel 4214 established around the perimeter of the inferior support plate 4202. In a particular embodiment, a portion of the sheath 4300 can be held within the inferior channel 4214 using an inferior retaining ring 4354.

As depicted in FIG. 43, the inferior support plate 4202 can include a bone growth promoting layer 4216 disposed, or otherwise deposited, on the inferior bearing surface 4206. In a particular embodiment, the bone growth promoting layer 4216 can include a biological factor that can promote bone on-growth or bone in-growth. For example, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, stem cells, or a combination thereof. Further, the stem cells can include bone marrow derived stem cells, lipo derived stem cells, or a combination thereof.

As depicted in FIG. 43, the nucleus 4300 can be generally toroid shaped. Further, the nucleus 4300 includes a core 4302 and an outer wear resistant layer 4304. In a particular embodiment, the core 4302 of the nucleus can be made from one or more biocompatible materials. For example, the biocompatible materials can be one or more polymer materials, described herein. Further, the outer wear resistant layer 4304 can be established by cross-linking the surface of the core 4302.

In a particular embodiment, depending on the type of material of which the core 4302 is comprised, the surface of the core 4302 can be cross-linked using a cross-linking agent. Acceptable cross-linking agents can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the core 4302 can be cross-linked by exposing the surface of the core 4302 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof; 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof. In a particular embodiment, the amount of cross-linking can vary depending on the type of material to be cross-linked, the time of exposure of the material to the cross-linking agent, the type of catalyst, etc. Also, in a particular embodiment, the surface of the core 4302 can be cross-linked to a depth of about five millimeters (5 mm) or less, such as about three millimeters (3 mm) or less. In this manner, the material underlying the wear resistant layer 4304 can exhibit the typical material properties associated with the uncross-linked material that comprises the core 4302.

Accordingly, the hardness of the wear resistant layer 4304 can be greater than the hardness of the underlying material. Further, the Young's modulus of the wear resistant layer 4304 can be greater than the Young's modulus of the underlying material. Also, the toughness of the wear resistant layer 4304 can be greater than the toughness of the underlying material.

Further, in a particular embodiment, the surface of the core 4302 can be cross-linked in such a fashion that the hardness of the wear resistant layer 4304 decreases from a maximum at or near the surface of the wear resistant layer 4304 to the underlying uncross-linked material of the core 4302. This can create a hardness gradient that substantially minimizes or eliminates an extreme change in hardness between the wear resistant layer 4304 and the core 4302. Further, the hardness gradient substantially minimizes or eliminates the chance that the wear resistant layer 4304 may delaminate from the core.

In another particular embodiment, the underlying material of the core 4302 may be cross-linked. However, in such a case, the mean or average cross-linking of the wear resistant layer 4304 may be greater than the underlying cross-linked material.

As illustrated in FIG. 43, the outer wear resistant layer 4304 of the nucleus 4300 can include a superior portion 4306 and an inferior portion 4308. In a particular embodiment, the superior portion 4306 of the outer wear resistant layer 4304 of the nucleus 4300 can be curved to match the curvature of the superior bearing surface 4106. Further, the superior portion 4306 of the outer wear resistant layer 4304 of the nucleus 4300 can slide relative to the superior bearing surface 4106 and can allow relative motion between the superior component 4100 and the nucleus 4300.

Also, in a particular embodiment, the inferior portion 4308 of the outer wear resistant layer 4304 of the nucleus 4300 can be curved to match the curvature of the inferior bearing surface 4206. Further, the inferior portion 4308 of the outer wear resistant layer 4304 of the nucleus 4300 can slide relative to the inferior bearing surface 4206 and can allow relative motion between the inferior component 4200 and the nucleus 4300.

In a particular embodiment, the entire outer surface of the nucleus 4300 can be cross-linked to establish the outer wear resistant layer 4304. Alternatively, a superior portion the outer surface, an inferior portion of the outer surface, or a combination thereof can be cross-linked.

The cross-linking agent can be introduced or applied at various points during manufacture of the prosthetic disc in order to accommodate various manufacturing parameters, including the desired degree of cross-linking at or near the surface. Alternatively, the cross-linking agent can be introduced or applied post-manufacture, yet prior to implantation (e.g., by surgical staff or the like). Alternatively, in certain embodiments, the cross-linking agent can be introduced or applied after implantation. Further, a cross-linking agent can be introduced or applied at various points between the beginning of manufacture and the end of the implantation procedure. Two or more different cross-linking agents can be introduced or applied at various points, as desired, to obtain the proper degree of cross-linking in the desired location(s). The cross-linking agent(s) can be provided along with all or a portion of the prosthetic disc in kit form for ease of use in the field.

Description of a Nucleus Implant

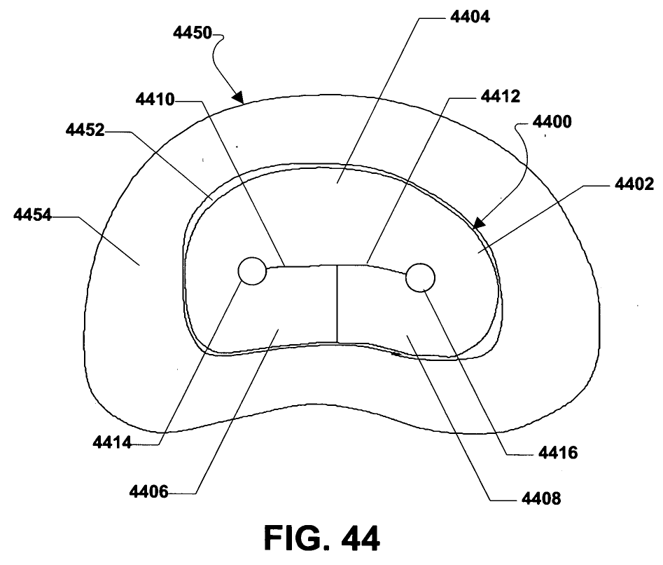

Referring to FIG. 44 through FIG. 47, an embodiment of a nucleus implant is shown and is designated 4400. As shown, the nucleus implant 4400 can include a load bearing elastic body 4402. The load bearing elastic body 4402 can include a central portion 4404. A first end 4406 and a second end 4408 can extend from the central portion 4404 of the load bearing elastic body 4402.

As depicted in FIG. 44, the first end 4406 of the load bearing elastic body 4402 can establish a first fold 4410 with respect to the central portion 4404 of the load bearing elastic body 4402. Further, the second end 4408 of the load bearing elastic body 4402 can establish a second fold 4412 with respect to the central portion 4404 of the load bearing elastic body 4402. In a particular embodiment, the ends 4406, 4408 of the load bearing elastic body 4402 can be folded toward each other relative to the central portion 4404 of the load bearing elastic body 4402. Also, when folded, the ends 4406, 4408 of the load bearing elastic body 4402 are parallel to the central portion 4404 of the load bearing elastic body 4402. Further, in a particular embodiment, the first fold 4410 can define a first aperture 4414 and the second fold 4412 can define a second aperture 4416. In a particular embodiment, the apertures 4414, 4416 are generally circular. However, the apertures 4414, 4416 can have any arcuate shape.

FIG. 44 indicates that the nucleus implant 4400 can be implanted within an intervertebral disc 4450 between a superior vertebra and an inferior vertebra. More specifically, the nucleus implant 4400 can be implanted within an intervertebral disc space 4452 established within the annulus fibrosus 4454 of the intervertebral disc 4450. The intervertebral disc space 4452 can be established by removing the nucleus pulposus (not shown) from within the annulus fibrosus 4454.

In a particular embodiment, the nucleus implant 4400 can provide shock-absorbing characteristics substantially similar to the shock absorbing characteristics provided by a natural nucleus pulposus. Additionally, in a particular embodiment, the nucleus implant 4400 can have a height that is sufficient to provide proper support and spacing between a superior vertebra and an inferior vertebra.

In a particular embodiment, the nucleus implant 4400 shown in FIG. 44 can have a shape memory and the nucleus implant 4400 can be configured to allow extensive short-term manual, or other, deformation without permanent deformation, cracks, tears, breakage or other damage, that may occur, for example, during placement of the implant into the intervertebral disc space 4452.

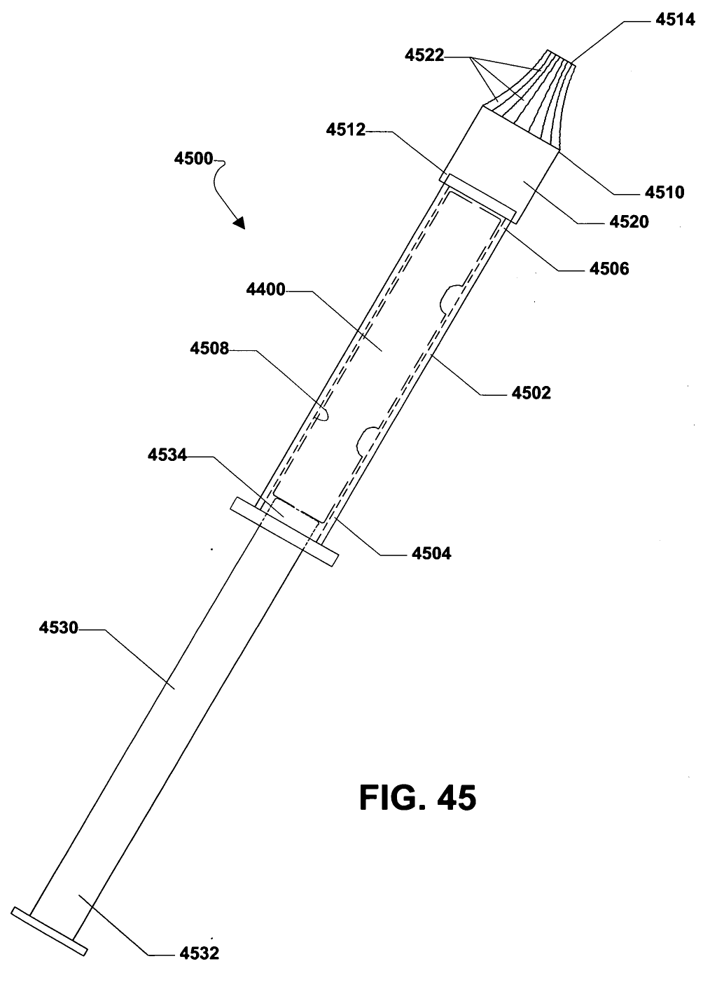

For example, the nucleus implant 4400 can be deformable, or otherwise configurable, e.g., manually, from a folded configuration, shown in FIG. 44, to a substantially straight configuration, shown in FIG. 45, in which the ends 4406, 4408 of the load bearing elastic body 4402 are substantially aligned with the central portion 4404 of the load bearing elastic body 4402. In a particular embodiment, when the nucleus implant 4400 the folded configuration, shown in FIG. 44, can be considered a relaxed state for the nucleus implant 4400. Also, the nucleus implant 4400 can be placed in the straight configuration for placement, or delivery into an intervertebral disc space within an annulus fibrosis.

In a particular embodiment, the nucleus implant 4400 can include a shape memory, and as such, the nucleus implant 4400 can automatically return to the folded, or relaxed, configuration from the straight configuration after force is no longer exerted on the nucleus implant 4400. Accordingly, the nucleus implant 4400 can provide improved handling and manipulation characteristics since the nucleus implant 4400 can be deformed, configured, or otherwise handled, by an individual without resulting in any breakage or other damage to the nucleus implant 4400.

Although the nucleus implant 4400 can have a wide variety of shapes, the nucleus implant 4400 when in the folded, or relaxed, configuration can conform to the shape of a natural nucleus pulposus. As such, the nucleus implant 4400 can be substantially elliptical when in the folded, or relaxed, configuration. In one or more alternative embodiments, the nucleus implant 4400, when folded, can be generally annular-shaped or otherwise shaped as required to conform to the intervertebral disc space within the annulus fibrosis. Moreover, when the nucleus implant 4400 is in an unfolded, or non-relaxed, configuration, such as the substantially straightened configuration, the nucleus implant 4400 can have a wide variety of shapes. For example, the nucleus implant 4400, when straightened, can have a generally elongated shape. Further, the nucleus implant 4400 can have a cross section that is: generally elliptical, generally circular, generally rectangular, generally square, generally triangular, generally trapezoidal, generally rhombic, generally quadrilateral, any generally polygonal shape, or any combination thereof.

Referring to FIG. 45, a nucleus delivery device is shown and is generally designated 4500. As illustrated in FIG. 45, the nucleus delivery device 4500 can include an elongated housing 4502 that can include a proximal end 4504 and a distal end 4506. The elongated housing 4502 can be hollow and can form an internal cavity 4508. As depicted in FIG. 45, the nucleus delivery device 4500 can also include a tip 4510 having a proximal end 4512 and a distal end 4514. In a particular embodiment, the proximal end 4512 of the tip 4510 can be affixed, or otherwise attached, to the distal end 4506 of the housing 4502.

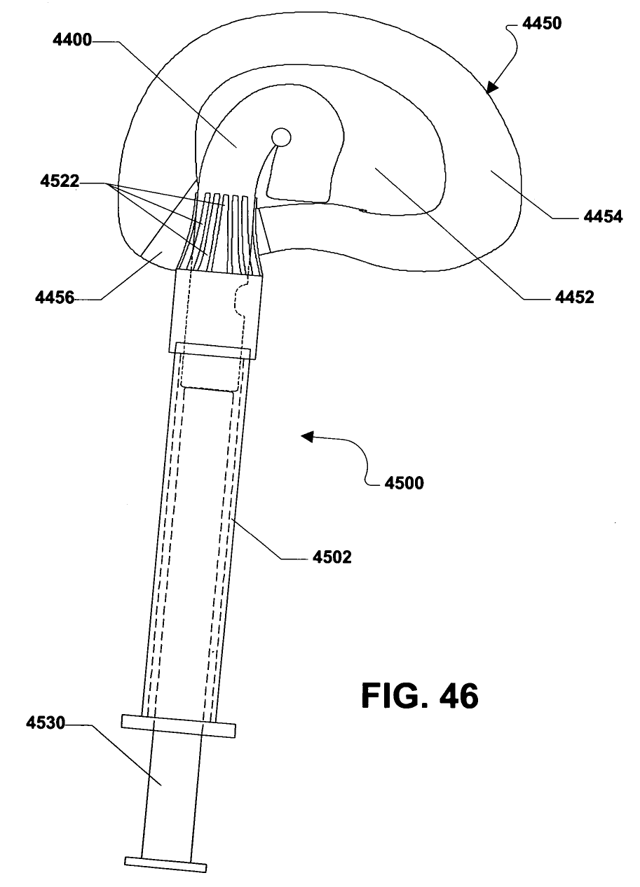

In a particular embodiment, the tip 4510 of the nucleus delivery device 4500 can include a generally hollow base 4520. Further, a plurality of movable members 4522 can be attached to the base 4520 of the tip 4510. The movable members 4522 are movable between a closed position, shown in FIG. 45, and an open position, shown in FIG. 46, as a nucleus implant is delivered using the nucleus delivery device 4500 as described below.

FIG. 45 further shows that the nucleus delivery device 4500 can include a generally elongated plunger 4530 that can include a proximal end 4532 and a distal end 4534. In a particular embodiment, the plunger 4530 can be sized and shaped to slidably fit within the housing 4502, e.g., within the cavity 4508 of the housing 4502.

As shown in FIG. 45 and FIG. 46, a nucleus implant, e.g., the nucleus implant 4400 shown in FIG. 44, can be disposed within the housing 4502, e.g., within the cavity 4508 of the housing 4502. Further, the plunger 4530 can slide within the cavity 4508, relative to the housing 4502, in order to force the nucleus implant 4400 from within the housing 4502 and into the intervertebral disc space 4452. As shown in FIG. 46, as the nucleus implant 4400 exits the nucleus delivery device 4500, the nucleus implant 4400 can move from the non-relaxed, straight configuration to the relaxed, folded configuration within the annulus fibrosis. Further, as the nucleus implant 4400 exits the nucleus delivery device 4500, the nucleus implant 4400 can cause the movable members 4522 to move to the open position, as shown in FIG. 46.

In a particular embodiment, the nucleus implant 4400 can be installed using a posterior surgical approach, as shown. Further, the nucleus implant 4400 can be installed through a posterior incision 4456 made within the annulus fibrosus 4454 of the intervertebral disc 4450. Alternatively, the nucleus implant 4400 can be installed using an anterior surgical approach, a lateral surgical approach, or any other surgical approach well known in the art.

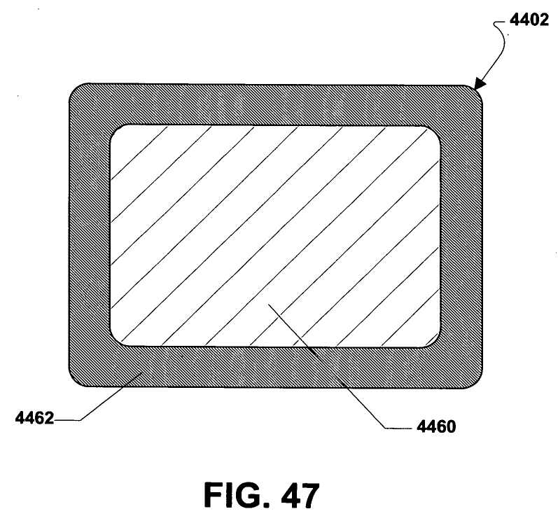

Referring to FIG. 47, the load bearing elastic body 4402 is illustrated in cross-section. As shown, the load bearing elastic body 4402 can include a core 4460 and an outer wear resistant layer 4462 that can surround the core 4460. In a particular embodiment, the core 4460 of the load bearing elastic body can be made from one or more biocompatible materials. For example, the biocompatible materials can be one or more polymer materials, described herein. Further, the outer wear resistant layer 4462 can be established by cross-linking the surface of the core 4460.

In a particular embodiment, depending on the type of material of which the core 4460 is comprised, the surface of the core 4460 can be cross-linked using a cross-linking agent. Acceptable cross-linking agents can include heat (thermal energy), various spectra or wavelengths of light, moisture, chemical agents/reagents, a radiation source (e.g., a thermal radiation source, a light radiation source, or another radiation source) or any combination of cross-linking agents. Further, the surface of the core 4460 can be cross-linked by exposing the surface of the core 4460 to a cross-linking agent in the presence of a catalyst. In various embodiments, the chemical cross-linking agents used can vary depending on the material to be cross-linked.

For example, for polyurethane materials suitable chemical cross-linking agents can include low molecular weight polyols or polyamines. Examples of such suitable chemical crosslinking agents can include, but are not limited to, trimethylolpropane, pentaerythritol, ISONOL® 93, trimethylolethane, triethanolamine, Jeffamines, 1,4-butanediamine, xylene diamine, diethylenetriamine, methylene dianiline, diethanolamine, or a combination thereof.

For silicone materials, suitable chemical cross-linking agents can include, but are not limited to, tetramethoxysilane, tetraethoxysilane, tetrapropoxysilane, tetrabutoxysilane, methyltrimethoxysilane, methyltriethoxysilane, vinyltrimethoxysilane, vinyltriethoxysilane, phenyltrimethoxysilane, phenyltriethoxysilane, 3-cyanopropyltrimethoxysilane, 3-cyanopropyltriethoxysilane, 3-(glycidyloxy)propyltriethoxysilane, 1,2-bis(trimethoxysilyl)ethane, 1,2-bis(triethoxysilyl)ethane, hexaethoxydisiloxane, or a combination thereof.

Additionally, for polyolefin materials, suitable chemical cross-linking agents can include an isocyanate, a polyol, a polyamine, or a combination thereof. The isocyanate can include 4,4′-diphenylmethane diisocyanate, polymeric 4,4′-diphenylmethane diisocyanate, carbodiimide-modified liquid 4,4′-diphenylmethane diisocyanate, 4,4′-dicyclohexylmethane diisocyanate, p-phenylene diisocyanate, toluene diisocyanate, isophoronediisocyanate, p-methylxylene diisocyanate, m-methylxylene diisocyanate, o-methylxylene diisocyanate, or a combination thereof. The polyol can include polyether polyols, hydroxy-terminated polybutadiene, polyester polyols, polycaprolactone polyols, polycarbonate polyols, or a combination thereof. Further, the polyamine can include 3,5-dimethylthio-2,4-toluenediamine or one or more isomers thereof, 3,5-diethyltoluene-2,4-diamine or one or more isomers thereof; 4,4′-bis-(sec-butylamino)-diphenylmethane; 1,4-bis-(sec-butylamino)-benzene, 4,4′-methylene-bis-(2-chloroaniline); 4,4′-methylene-bis-(3-chloro-2,6-diethylaniline); trimethylene glycol-di-p-aminobenzoate; polytetramethyleneoxide-di-p-aminobenzoate; N,N′-dialkyldiamino diphenyl methane; p, p′-methylene dianiline; phenylenediamine; 4,4′-methylene-bis-(2-chloroaniline); 4,4′-methylene-bis-(2,6-diethylaniline); 4,4′-diamino-3,3′-diethyl-5,5′-dimethyl-diphenylmethane; 2,2′,3,3′-tetrachloro diamino diphenylmethane; 4,4′-methylene-bis-(3-chloro-2,6-diethylaniline); or a combination thereof.

In another embodiment, the chemical cross-linking agent is a polyol curing agent. The polyol curing agent may include ethylene glycol; diethylene glycol; polyethylene glycol; propylene glycol; polypropylene glycol; lower molecular weight polytetramethylene ether glycol; 1,3-bis(2-hydroxyethoxy)benzene; 1,3-bis-[2-(2-hydroxyethoxy)ethoxy]benzene; 1,3-bis-{2-[2-(2-hydroxyethoxy)ethoxy]ethoxy}benzene; 1,4-butanediol; 1,5-pentanediol; 1,6-hexanediol; resorcinol-di-(β-hydroxyethyl)ether; hydroquinone-di-(β-hydroxyethyl)ether; trimethylol propane, and mixtures thereof.

In a particular embodiment, the amount of cross-linking can vary depending on the type of material to be cross-linked, the time of exposure of the material to the cross-linking agent, the type of catalyst, etc. Also, in a particular embodiment, the surface of the core 4460 can be cross-linked to a depth of about five millimeters (5 mm) or less, such as about three millimeters (3 mm) or less. In this manner, the material underlying the wear resistant layer 4462 can exhibit the typical material properties associated with the uncross-linked material that comprises the core 4460.

Accordingly, the hardness of the wear resistant layer 4462 can be greater than the hardness of the underlying material. Further, the Young's modulus of the wear resistant layer 4462 can be greater than the Young's modulus of the underlying material. Also, the toughness of the wear resistant layer 4462 can be greater than the toughness of the underlying material.

Further, in a particular embodiment, the surface of the core 4460 can be cross-linked in such a fashion that the hardness of the wear resistant layer 4462 decreases from a maximum at or near the surface of the wear resistant layer 4462 to the underlying uncross-linked material of the core 4460. This can create a hardness gradient that substantially minimizes or eliminates an extreme change in hardness between the wear resistant layer 4462 and the core 4460. Further, the hardness gradient substantially minimizes or eliminates the chance that the wear resistant layer 4462 may delaminate from the core.

In another particular embodiment, the underlying material of the core 4460 may be cross-linked. However, in such a case, the mean or average cross-linking of the wear resistant layer 4462 may be greater than the underlying cross-linked material.

The cross-linking agent can be introduced or applied at various points during manufacture of the implant in order to accommodate various manufacturing parameters, including the desired degree of cross-linking at or near the surface. Alternatively, the cross-linking agent can be introduced or applied post-manufacture, yet prior to implantation (e.g., by surgical staff or the like). Alternatively, in certain embodiments, the cross-linking agent can be introduced or applied after implantation. Further, a cross-linking agent can be introduced or applied at various points between the beginning of manufacture and the end of the implantation procedure. Two or more different cross-linking agents can be introduced or applied at various points, as desired, to obtain the proper degree of cross-linking in the desired location(s). The cross-linking agent(s) can be provided along with all or a portion of the implant in kit form for ease of use in the field.

### CONCLUSION

With the configuration of structure described above, the intervertebral prosthetic disc or nucleus implant according to one or more of the embodiments provides a device that may be implanted to replace at least a portion of a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion there between.

In a particular embodiment, the wear resistant layers provided by one or more of the intervertebral prosthetic discs described herein can limit the wear of the moving components caused by motion and friction. Further, the wear resistant layers provided by one or more of the intervertebral prosthetic discs described herein can increase the life of an intervertebral prosthetic disc. Accordingly, the time before the intervertebral prosthetic disc may need to be replaced can be substantially increased. Further, the wear resistant layers described herein can reduce the occurrence and amount of wear debris, which could otherwise produce undesired or deleterious effects on collateral systems.

In alternative embodiments, other intervertebral implants having bearing surfaces or articulating surfaces may be cross-linked as described herein to increase the wear resistance of such intervertebral implants. Such implants can include implants of varying shapes and can include a sphere, a hemisphere, a solid ellipse, a cube, a cylinder, a pyramid, a prism, a rectangular solid shape, a cone, a frustum, or a combination thereof. Further, each of the various implants can include at least one bearing surface or articulating surface that can be cross-linked greater than a core. As stated above, the core may or may not be cross-linked.

Additional implant structures may also be cross-linked as described herein. For example, a component may include a polymeric rod within a collar. The polymeric rod may have its surface cross-linked to prevent against wear caused by relative motion between the polymeric rod and the collar.

The above-disclosed subject matter is to be considered illustrative, and not restrictive, and the appended claims are intended to cover all such modifications, enhancements, and other embodiments that fall within the true spirit and scope of the present invention. For example, it is noted that the components in the exemplary embodiments described herein are referred to as “superior” and “inferior” for illustrative purposes only and that one or more of the features described as part of or attached to a respective half may be provided as part of or attached to the other half in addition or in the alternative. Thus, to the maximum extent allowed by law, the scope of the present invention is to be determined by the broadest permissible interpretation of the following claims and their equivalents, and shall not be restricted or limited by the foregoing detailed description.

1. An intervertebral prosthetic disc configured to be installed within an intervertebral space between a superior vertebra and an inferior vertebra, the intervertebral prosthetic disc comprising:
an inferior component having a depression formed therein; and
a superior component having a projection extending therefrom, wherein the projection is configured to movably engage the depression and allow relative motion between the inferior component and the superior component and wherein the projection includes a superior wear resistant layer comprising a cross-linked polymer and configured to engage the depression.

2. The intervertebral prosthetic disc of claim 1, wherein the superior wear resistant layer is formed by cross-linking a surface of the projection.

3. The intervertebral prosthetic disc of claim 2, wherein the surface of the projection is cross-linked to a depth not greater than three millimeters.

4. The intervertebral prosthetic disc of claim 1, wherein a hardness of the superior wear resistant layer decreases with depth.

5. The intervertebral prosthetic disc of claim 1, wherein a Young's modulus of the superior wear resistant layer decreases with depth.

6. The intervertebral prosthetic disc of claim 1, wherein a toughness of the superior wear resistant layer decreases with depth.

7. The intervertebral prosthetic disc of claim 1, wherein the inferior component further comprises an inferior wear resistant layer within the depression wherein the inferior wear resistant layer is configured to engage the superior wear resistant layer.

8. The intervertebral prosthetic disc of claim 7, wherein the inferior wear resistant layer is formed by cross-linking a surface of the depression.

9. The intervertebral prosthetic disc of claim 8, wherein the surface of the projection is cross-linked to a depth not greater than three millimeters.

10. The intervertebral prosthetic disc of claim 9, wherein a hardness of the superior wear resistant layer decreases with depth.

11. The intervertebral prosthetic disc of claim 7, wherein a Young's modulus of the superior wear resistant layer decreases with depth.

12. The intervertebral prosthetic disc of claim 7, wherein a toughness of the superior wear resistant layer decreases with depth

13. The intervertebral prosthetic disc of claim 1, wherein the superior component further comprises a superior bracket extending therefrom, wherein the superior bracket is configured to be attached to the superior vertebra.

14. The intervertebral prosthetic disc of claim 1, wherein the inferior component further comprises an inferior bracket extending therefrom, wherein the inferior bracket is configured to be attached to the inferior vertebra.

15. The intervertebral prosthetic disc of claim 1, wherein the superior component, the inferior component, or a combination thereof is made from a biocompatible material.

16. The intervertebral prosthetic disc of claim 15, wherein the biocompatible material is a polymer.

17. The intervertebral prosthetic disc of claim 16, wherein the polymer comprises polyurethane, a polyolefin, a polyaryletherketon (PAEK), a silicone, a hydrogel, or a combination thereof.

18. The intervertebral prosthetic disc of claim 17, wherein the polyolefin comprises polypropylene, polyethylene, halogenated polyolefin, fluoropolyolefin, polybutadiene, or a combination thereof.

19. The intervertebral prosthetic disc of claim 17, wherein the polyaryletherketon (PAEK) comprises polyetherketone (PEK), polyetheretherketone (PEEK), polyetherketoneketone (PEKK), polyetherketoneetherketoneketone (PEKEKK), or a combination thereof.

20. An intervertebral prosthetic disc configured to be installed within an intervertebral space between a superior vertebra and an inferior vertebra, the intervertebral prosthetic disc comprising:
an inferior component having an inferior depression formed therein;
a superior component having a superior depression formed therein; and
a nucleus disposed between the inferior component and the superior component, wherein the nucleus includes a superior wear resistant layer and an inferior wear resistant layer, wherein the superior wear resistant layer of the nucleus comprising a cross-linked polymer and is configured to movably engage the superior depression and wherein the inferior wear resistant layer of the nucleus is configured to movably engage the inferior depression.

21-29. (canceled)

30. The intervertebral prosthetic disc of claim 26, wherein a Young's modulus of the superior wear resistant layer of the superior component decreases with depth.

31-44. (canceled)

45. An intervertebral prosthetic disc configured to be installed within an intervertebral space between a superior vertebra and an inferior vertebra, the intervertebral prosthetic disc comprising:
an inferior component;
a superior component; and
a generally toroidal nucleus disposed between the inferior component and the superior component, wherein the nucleus includes a core and an outer wear resistant layer on the core, wherein the outer wear resistant layer of the core comprising a cross-linked polymer and is configured to movably engage the inferior component and the superior component.

46-53. (canceled)

54. A nucleus implant configured to be installed within an intervertebral space within an intervertebral disc comprising:
a load bearing elastic body movable between a folded configuration and a substantially straight configuration, wherein the load bearing elastic body has a core and an outer wear resistant layer around the core, wherein the outer wear resistant layer comprises a cross-linked polymer.

55-60. (canceled)

61. An intervertebral prosthetic disc configured to be installed within an intervertebral space between a superior vertebra and an inferior vertebra, the intervertebral prosthetic disc comprising:
a first polymer component having a main body and a wear surface, wherein the wear surface exhibits a higher degree of cross-linking than a portion of the main body.

62-64. (canceled)

65. A spinal implant configured to be installed between a superior vertebra and an inferior vertebra, the spinal implant comprising:
a polymeric component having a surface, wherein the surface of the polymeric component is cross-linked greater than an underlying material.

66-70. (canceled)
